SEQUENCE LISTING (538 sequences)

SEQ ID NO: 1 - protein, 736 aa, AAV-1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, AAV-2 [sequence omitted]

SEQ ID NO: 3 - protein, 736 aa, AAV-3b
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 734 aa, AAV-4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 5 - protein, 724 aa, AAV-5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - protein, 736 aa, AAV-6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - protein, 737 aa, AAV-7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 8 - protein, 738 aa, AAV-8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 9 - protein, 736 aa, AAV-9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 738 aa, AAV-10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 11 - protein, 700 aa, AAV-11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTS

SEQ ID NO: 12 - protein, 709 aa, AAVbovine
GDPVNFADEVAREHDLSYQKQLEAGDNPYLKYNHADAEFQEKLASDTSFGGNLGKAVFQAKKRILEPLGLVETPDKTAPAAKKRPLEQSPQEPDSSSGVGKKGKQPARKRLNFDDEPGAGDGPPPEGPSSGAMSTETEMRAAAGGNGGDAGQGAEGVGNASGDWHCDSTWSESHVTTTSTRTWVLPTYNNHLYLRLGSSNASDTFNGFSTPWGYFDFNRFHCHFSPRDWQRLINNHWGLRPKSMQVRIFNIQVKEVTTSNGETTVSNNLTSTVQIFADSTYELPYVMDAGQEGSLPPFPNDVFMVPQYGYCGLVTGGSSQNQTDRNAFYCLEYFPSQMLRTGNNFEMVYKFENVPFHSMYAHSQSLDRLMNPLLDQYLWELQSTTSGGTLNQGNSATNFAKLTKTNFSGYRKNWLPGPMMKQQRFSKTASQNYKIPQGRNNSLLHYETRTTLDGRWSNFAPGTAMATAANDATDFSQAQLIFAGPNITGNTTTDANNLMFTSEDELRATNPRDTDLFGHLATNQQNATTVPTVDDVDGVGVYPGMVWQDRDIYYQGPIWAKIPHTDGHFHPSPLIGGFGLKSPPPQIFIKNTPVPANPATTFSPARINSFITQYSTGQVAVKIEWEIQKERSKRWNPEVQFTSNYGAQDSLLWAPDNAGAYKEPRAIGSRYLTNHLNYGNQSSMLWAPDTTGKYTEPRVIGSRYLTNHL

SEQ ID NO: 13 - protein, 7 aa, artificial
  peptide
KGGALGG

SEQ ID NO: 14 - protein, 7 aa, artificial
  peptide
ASTGPHT

SEQ ID NO: 15 - protein, 7 aa, artificial
  peptide
TSPSPSR

SEQ ID NO: 16 - protein, 7 aa, artificial
  peptide
NNPGAPR

SEQ ID NO: 17 - protein, 7 aa, artificial
  peptide
GVSPERR

SEQ ID NO: 18 - protein, 7 aa, artificial
  peptide
WHAPTHG

SEQ ID NO: 19 - protein, 7 aa, artificial
  peptide
NAQPPSR

SEQ ID NO: 20 - protein, 7 aa, artificial
  peptide
HAAGLPK

SEQ ID NO: 21 - protein, 7 aa, artificial
  peptide
PANQSVR

SEQ ID NO: 22 - protein, 7 aa, artificial
  peptide
GSNATAS

SEQ ID NO: 23 - protein, 7 aa, artificial
  peptide
GAHRSDS

SEQ ID NO: 24 - protein, 7 aa, artificial
  peptide
ANYQGPA

SEQ ID NO: 25 - protein, 7 aa, artificial
  peptide
NHTAATW

SEQ ID NO: 26 - protein, 7 aa, artificial
  peptide
TNHASAA

SEQ ID NO: 27 - protein, 7 aa, artificial
  peptide
NYPGAHS

SEQ ID NO: 28 - protein, 7 aa, artificial
  peptide
IGTNSPV

SEQ ID NO: 29 - protein, 7 aa, artificial
  peptide
AQYSAAV

SEQ ID NO: 30 - protein, 7 aa, artificial
  peptide
SYGQSAG

SEQ ID NO: 31 - protein, 7 aa, artificial
  peptide
AHGGSIP

SEQ ID NO: 32 - protein, 7 aa, artificial
  peptide
YQSGSLP

SEQ ID NO: 33 - protein, 7 aa, artificial
  peptide
DSQHTQH

SEQ ID NO: 34 - protein, 7 aa, artificial
  peptide
GSDRVST

SEQ ID NO: 35 - protein, 7 aa, artificial
  peptide
TSNRAAT

SEQ ID NO: 36 - protein, 7 aa, artificial
  peptide
GGTTLAH

SEQ ID NO: 37 - protein, 7 aa, artificial
  peptide
SSPNHVA

SEQ ID NO: 38 - protein, 7 aa, artificial
  peptide
GNARPPD

SEQ ID NO: 39 - protein, 7 aa, artificial
  peptide
PGQTRDS

SEQ ID NO: 40 - protein, 7 aa, artificial
  peptide
ENATLRT

SEQ ID NO: 41 - protein, 7 aa, artificial
  peptide
HTRTSQE

SEQ ID NO: 42 - protein, 7 aa, artificial
  peptide
PVGSPSR

SEQ ID NO: 43 - protein, 7 aa, artificial
  peptide
SSGTTRD

SEQ ID NO: 44 - protein, 7 aa, artificial
  peptide
DDGRPSR

SEQ ID NO: 45 - protein, 7 aa, artificial
  peptide
AAYGAQS

SEQ ID NO: 46 - protein, 7 aa, artificial
  peptide
REGSPGR

SEQ ID NO: 47 - protein, 7 aa, artificial
  peptide
NKTGASP

SEQ ID NO: 48 - protein, 7 aa, artificial
  peptide
KASGQTV

SEQ ID NO: 49 - protein, 7 aa, artificial
  peptide
RDTTTGA

SEQ ID NO: 50 - protein, 7 aa, artificial
  peptide
AYGPNAG

SEQ ID NO: 51 - protein, 7 aa, artificial
  peptide
DSDGPRP

SEQ ID NO: 52 - protein, 7 aa, artificial
  peptide
GSSATAS

SEQ ID NO: 53 - protein, 7 aa, artificial
  peptide
TSAGSPR

SEQ ID NO: 54 - protein, 7 aa, artificial
  peptide
GPAHTSS

SEQ ID NO: 55 - protein, 7 aa, artificial
  peptide
NAAGTPR

SEQ ID NO: 56 - protein, 7 aa, artificial
  peptide
NRTALGS

SEQ ID NO: 57 - protein, 7 aa, artificial
  peptide
KSHSASP

SEQ ID NO: 58 - protein, 7 aa, artificial
  peptide
RGHTAPI

SEQ ID NO: 59 - protein, 7 aa, artificial
  peptide
PTSGAPK

SEQ ID NO: 60 - protein, 7 aa, artificial
  peptide
GNRVQST

SEQ ID NO: 61 - protein, 7 aa, artificial
  peptide
NTSLTAR

SEQ ID NO: 62 - protein, 7 aa, artificial
  peptide
RDRATSP

SEQ ID NO: 63 - protein, 7 aa, artificial
  peptide
GAERTYS

SEQ ID NO: 64 - protein, 7 aa, artificial
  peptide
ARGSVSP

SEQ ID NO: 65 - protein, 7 aa, artificial
  peprtide
TGRSSGD

SEQ ID NO: 66 - protein, 7 aa, artificial
  peptide
HPARPNA

SEQ ID NO: 67 - protein, 7 aa, artificial
  peptide
SDHLHPR

SEQ ID NO: 68 - protein, 7 aa, artificial
  peptide
TVLSSQR

SEQ ID NO: 69 - protein, 7 aa, artificial
  peptide
VSSSSRP

SEQ ID NO: 70 - protein, 7 aa, artificial
  peptide
TGSSKGV

SEQ ID NO: 71 - protein, 7 aa, artificial
  peptide
SPGGPAR

SEQ ID NO: 72 - protein, 7 aa, artificial
  peptide
GRSEQIR

SEQ ID NO: 73 - protein, 7 aa, artificial
  peptide
VTARPEG

SEQ ID NO: 74 - protein, 7 aa, artificial
  peptide
RDPSPGR

SEQ ID NO: 75 - protein, 7 aa, artificial
  peptide
AASGVAR

SEQ ID NO: 76 - protein, 7 aa, artificial
  peptide
TSARSVP

SEQ ID NO: 77 - protein, 7 aa, artificial
  peptide
ARGGSIP

SEQ ID NO: 78 - protein, 7 aa, artificial
  peptide
RAAGQVA

SEQ ID NO: 79 - protein, 7 aa, artificial
  peptide
TSQQRIP

SEQ ID NO: 80 - protein, 7 aa, artificial
  peptide
NSARPNS

SEQ ID NO: 81 - protein, 7 aa, artificial
  peptide
PVGSPGR

SEQ ID NO: 82 - protein, 7 aa, artificial
  peptide
TQSPPGR

SEQ ID NO: 83 - protein, 7 aa, artificial
  peptride
GRRDSPV

SEQ ID NO: 84 - protein, 7 aa, artificial
  peptide
NGSPVGR

SEQ ID NO: 85 - protein, 7 aa, artificial
  peptide
KVSGHQA

SEQ ID NO: 86 - protein, 7 aa, artificial
  peptide
ARSQVHA

SEQ ID NO: 87 - protein, 7 aa, artificial
  peptide
ARTTHSP

SEQ ID NO: 88 - protein, 7 aa, artificial
  peprtide
NLSRASP

SEQ ID NO: 89 - protein, 7 aa, artificial
  peptide
NTPQSAR

SEQ ID NO: 90 - protein, 7 aa, artificial
  peptide
GRPGSGY

SEQ ID NO: 91 - protein, 7 aa, artificial
  peptide
VPSAPQR

SEQ ID NO: 92 - protein, 7 aa, artificial
  peptide
APANLPR

SEQ ID NO: 93 - protein, 7 aa, artificial
  peptide
LGRPAGP

SEQ ID NO: 94 - protein, 7 aa, artificial
  peptide
VLGTKGA

SEQ ID NO: 95 - protein, 7 aa, artificial
  peptide
GRPEQIR

SEQ ID NO: 96 - protein, 7 aa, artificial
  peptide
GLAGVVR

SEQ ID NO: 97 - protein, 7 aa, artificial
  peptide
AHRSTGT

SEQ ID NO: 98 - protein, 7 aa, artificial
  peptide
GTSPANR

SEQ ID NO: 99 - protein, 7 aa, artificial
  peptide
NRPNAAS

SEQ ID NO: 100 - protein, 7 aa, artificial
  peptide
ARSGSGA

SEQ ID NO: 101 - protein, 7 aa, artificial
  peptide#
RHSSTPV

SEQ ID NO: 102 - protein, 7 aa, artificial
  peptide
NSPLGTR

SEQ ID NO: 103 - protein, 7 aa, artificial
  peptide
NTPSPAR

SEQ ID NO: 104 - protein, 7 aa, artificial
  peptide
NSPAAGR

SEQ ID NO: 105 - protein, 7 aa, artificial
  peptide
SNRTSSV

SEQ ID NO: 106 - protein, 7 aa, artificial
  peptide
SVRGGTP

SEQ ID NO: 107 - protein, 7 aa, artificial
  peptide
SPADSRV

SEQ ID NO: 108 - protein, 7 aa, artificial
  peptide
STRGQGH

SEQ ID NO: 109 - protein, 7 aa, artificial
  peptide
VDSGLSG

SEQ ID NO: 110 - protein, 7 aa, artificial
  peptide
GDGSFSG

SEQ ID NO: 111 - protein, 7 aa, artificial
  peptide
VSGPPHT

SEQ ID NO: 112 - protein, 7 aa, artificial
  peptide
RHGPQHP

SEQ ID NO: 113 - protein, 7 aa, artificial
  peptide
RDRAASP

SEQ ID NO: 114 - protein, 7 aa, artificial
  peptide
GDRATSP

SEQ ID NO: 115 - protein, 7 aa, artificial
  peptide
RGRATSP

SEQ ID NO: 116 - protein, 7 aa, artificial
  peptide
RDRVTSP

SEQ ID NO: 117 - protein, 7 aa, artificial
  peptide
PVLNPPP

SEQ ID NO: 118 - protein, 7 aa, artificial
  peptride
SFQRASP

SEQ ID NO: 119 - protein, 7 aa, artificial
  peptide
RDRATTP

SEQ ID NO: 120 - protein, 7 aa, artificial
  peptide
QSRLSTQ

SEQ ID NO: 121 - protein, 7 aa, artificial
  peptide
SSGGGTP

SEQ ID NO: 122 - protein, 7 aa, artificial
  peptide
KGEALGG

SEQ ID NO: 123 - protein, 7 aa, artificial
  peptide
SVRGAPP

SEQ ID NO: 124 - protein, 7 aa, artificial
  peptide
DHDSPSV

SEQ ID NO: 125 - protein, 7 aa, artificial
  peptide
RDQATSP

SEQ ID NO: 126 - protein, 7 aa, artificial
  peptide
RDRATSS

SEQ ID NO: 127 - protein, 7 aa, artificial
  peptide
GKPPRTE

SEQ ID NO: 128 - protein, 7 aa, artificial
  peptide
AHRGLQV

SEQ ID NO: 129 - protein, 7 aa, artificial
  peptide
VASLAAR

SEQ ID NO: 130 - protein, 7 aa, artificial
  peptide
RDRTTSP

SEQ ID NO: 131 - protein, 7 aa, artificial
  peptide
RDWATSP

SEQ ID NO: 132 - protein, 7 aa, artificial
  peprtide
RNRATSP

SEQ ID NO: 133 - protein, 7 aa, artificial
  peptide
RDRATSL

SEQ ID NO: 134 - protein, 7 aa, artificial
  peptide
KDRATSP

SEQ ID NO: 135 - protein, 7 aa, artificial
  peptide
ARYSAAV

SEQ ID NO: 136 - protein, 7 aa, artificial
  peptide
GAHRSVS

SEQ ID NO: 137 - protein, 7 aa, artificial
  peptide
WDRATSP

SEQ ID NO: 138 - protein, 7 aa, artificial
  peptide
ERPYQAG

SEQ ID NO: 139 - protein, 7 aa, artificial
  peptide
NAPGNLR

SEQ ID NO: 140 - protein, 7 aa, artificial
  peptide
NNPLPQR

SEQ ID NO: 141 - protein, 7 aa, artificial
  peptide
ARPTVPY

SEQ ID NO: 142 - protein, 7 aa, artificial
  peptide
RSGAAVA

SEQ ID NO: 143 - protein, 7 aa, artificial
  peptide
NTPNPLR

SEQ ID NO: 144 - protein, 7 aa, artificial
  peptide
GGTTLGR

SEQ ID NO: 145 - protein, 7 aa, artificial
  peptide
ASTGLPR

SEQ ID NO: 146 - protein, 7 aa, artificial
  peptide
ATGVIQR

SEQ ID NO: 147 - protein, 7 aa, artificial
  peptide
NSAALGR

SEQ ID NO: 148 - protein, 7 aa, artificial
  peptide
GEPQPRF

SEQ ID NO: 149 - protein, 7 aa, artificial
  peptide
GHSARSI

SEQ ID NO: 150 - protein, 7 aa, artificial
  peptide
VSSSTPR

SEQ ID NO: 151 - protein, 7 aa, artificial
  peptide
NQPNPVR

SEQ ID NO: 152 - protein, 7 aa, artificial
  peptide
PSGGPPR

SEQ ID NO: 153 - protein, 7 aa, artificial
  peptide
ARDQLPR

SEQ ID NO: 154 - protein, 7 aa, artificial
  peptide
RGSHPNI

SEQ ID NO: 155 - protein, 7 aa, artificial
  peptide
NRASNTS

SEQ ID NO: 156 - protein, 7 aa, artificial
  peptide
VNAVPGR

SEQ ID NO: 157 - protein, 7 aa, artificial
  peptide
PANTPGR

SEQ ID NO: 158 - protein, 7 aa, artificial
  peptide
ISSSTAR

SEQ ID NO: 159 - protein, 7 aa, artificial
  peptide
ARAGRDL

SEQ ID NO: 160 - protein, 7 aa, artificial
  peptide
RGGEVPR

SEQ ID NO: 161 - protein, 7 aa, artificial
  peptide
VNLVPSR

SEQ ID NO: 162 - protein, 7 aa, artificial
  peptide
LARNQSG

SEQ ID NO: 163 - protein, 7 aa, artificial
  peptide
SSSGSPR

SEQ ID NO: 164 - protein, 7 aa, artificial
  peptide
SPGINLR

SEQ ID NO: 165 - protein, 7 aa, artificial
  peptide
AVAGSPR

SEQ ID NO: 166 - protein, 7 aa, artificial
  peptide
RSPDSAR

SEQ ID NO: 167 - protein, 7 aa, artificial
  peptide
NQPVPNR

SEQ ID NO: 168 - protein, 7 aa, artificial
  peptide
PSSPVAR

SEQ ID NO: 169 - protein, 7 aa, artificial
  peptide
KDAQLGR

SEQ ID NO: 170 - protein, 7 aa, artificial
  peptide
VTSSSGK

SEQ ID NO: 171 - protein, 7 aa, artificial
  peptide
AANMTAR

SEQ ID NO: 172 - protein, 7 aa, artificial
  peptide
SSINPPR

SEQ ID NO: 173 - protein, 7 aa, artificial
  peptide
AYAGTPK

SEQ ID NO: 174 - protein, 7 aa, artificial
  peptide
RGSSPVR

SEQ ID NO: 175 - protein, 7 aa, artificial
  peptide
GGGLTPR

SEQ ID NO: 176 - protein, 7 aa, artificial
  peptide
GASSVSR

SEQ ID NO: 177 - protein, 7 aa, artificial
  peptide
VHLSPGR

SEQ ID NO: 178 - protein, 7 aa, artificial
  peptide
NSPNVAR

SEQ ID NO: 179 - protein, 7 aa, artificial
  peptide
GGRDQLR

SEQ ID NO: 180 - protein, 7 aa, artificial
  peptide
AAQGMPK

SEQ ID NO: 181 - protein, 7 aa, artificial
  peptide
RRDLDVR

SEQ ID NO: 182 - protein, 7 aa, artificial
  peptide
VTSTSAR

SEQ ID NO: 183 - protein, 7 aa, artificial
  peptide
RHGSPGS

SEQ ID NO: 184 - protein, 7 aa, artificial
  peptide
NSPSGPR

SEQ ID NO: 185 - protein, 7 aa, artificial
  peptide
RDGITSR

SEQ ID NO: 186 - protein, 7 aa, artificial
  peptide
SNAPSAR

SEQ ID NO: 187 - protein, 7 aa, artificial
  peptide
VMNGQLR

SEQ ID NO: 188 - protein, 7 aa, artificial
  peptide
YSAGSPK

SEQ ID NO: 189 - protein, 7 aa, artificial
  peptide
SDNHTAR

SEQ ID NO: 190 - protein, 7 aa, artificial
  peptide
RDRAGPP

SEQ ID NO: 191 - protein, 7 aa, artificial
  peptide
RVRATSP

SEQ ID NO: 192 - protein, 7 aa, artificial
  peptide
STRGHGA

SEQ ID NO: 193 - protein, 7 aa, artificial
  peptide
STRGRGA

SEQ ID NO: 194 - protein, 7 aa, artificial
  peptide
NDVRAPP

SEQ ID NO: 195 - protein, 7 aa, artificial
  peptide
SNGGSPV

SEQ ID NO: 196 - protein, 7 aa, artificial
  peptide
MHASPSG

SEQ ID NO: 197 - protein, 7 aa, artificial
  peptide
AASHPNQ

SEQ ID NO: 198 - protein, 7 aa, artificial
  peptide
DTGSHAL

SEQ ID NO: 199 - protein, 7 aa, artificial
  peptide
SPSDRFS

SEQ ID NO: 200 - protein, 7 aa, artificial
  peptide
GPNATAS

SEQ ID NO: 201 - protein, 7 aa, artificial
  peptide
GGPGYTA

SEQ ID NO: 202 - protein, 7 aa, artificial
  peptide
GSDATAS

SEQ ID NO: 203 - protein, 7 aa, artificial
  peptide
AQYSAAA

SEQ ID NO: 204 - protein, 7 aa, artificial
  peptide
VPTLRPA

SEQ ID NO: 205 - protein, 7 aa, artificial
  peptide
AGTGIPV

SEQ ID NO: 206 - protein, 7 aa, artificial
  peptide
GSNATAG

SEQ ID NO: 207 - protein, 7 aa, artificial
  peptide
GPANSSH

SEQ ID NO: 208 - protein, 7 aa, artificial
  peptide
AARSTDL

SEQ ID NO: 209 - protein, 7 aa, artificial
  peptide
SHGGLSE

SEQ ID NO: 210 - protein, 7 aa, artificial
  peptide
GQAMSGG

SEQ ID NO: 211 - protein, 7 aa, artificial
  peptide
GTNATAS

SEQ ID NO: 212 - protein, 7 aa, artificial
  peptide
FTAANNQ

SEQ ID NO: 213 - protein, 7 aa, artificial
  peptide
SSNATAS

SEQ ID NO: 214 - protein, 7 aa, artificial
  peptide
GSNTTAS

SEQ ID NO: 215 - protein, 7 aa, artificial
  peptide
VSPNHAA

SEQ ID NO: 216 - protein, 7 aa, artificial
  peptide
GSNAAAS

SEQ ID NO: 217 - protein, 7 aa, artificial
  peptide
NVRPHPA

SEQ ID NO: 218 - protein, 7 aa, artificial
  peptide
NSERSVQ

SEQ ID NO: 219 - protein, 7 aa, artificial
  peptide
TVGRDSV

SEQ ID NO: 220 - protein, 7 aa, artificial
  peptide
PSDSTDR

SEQ ID NO: 221 - protein, 7 aa, artificial
  peptide
PANTPPR

SEQ ID NO: 222 - protein, 7 aa, artificial
  peptide
GANRPGL

SEQ ID NO: 223 - protein, 7 aa, artificial
  peptide
TNHAGAA

SEQ ID NO: 224 - protein, 7 aa, artificial
  peptide
ASTGPRT

SEQ ID NO: 225 - protein, 7 aa, artificial
  peptide
SSYAPSS

SEQ ID NO: 226 - protein, 7 aa, artificial
  peptide
AQPPQPP

SEQ ID NO: 227 - protein, 7 aa, artificial
  peptide
APTGPHT

SEQ ID NO: 228 - protein, 7 aa, artificial
  peptide
AHLSGTS

SEQ ID NO: 229 - protein, 7 aa, artificial
  peptide
TRSGDSS

SEQ ID NO: 230 - protein, 7 aa, artificial
  peptide
RTGRTSP

SEQ ID NO: 231 - protein, 7 aa, artificial
  peptide
RTGRDSP

SEQ ID NO: 232 - protein, 7 aa, artificial
  peptide
DDFQRAR

SEQ ID NO: 233 - protein, 7 aa, artificial
  peptide
RDRADSP

SEQ ID NO: 234 - protein, 7 aa, artificial
  peptide
RDRAISP

SEQ ID NO: 235 - protein, 7 aa, artificial
  peptide
RDRRTSP

SEQ ID NO: 236 - protein, 7 aa, artificial
  peptide
GARRSDS

SEQ ID NO: 237 - protein, 7 aa, artificial
  peptide
RTGGTSP

SEQ ID NO: 238 - protein, 7 aa, artificial
  peptide
RTGATSP

SEQ ID NO: 239 - protein, 7 aa, artificial
  peptide
GAHRPDS

SEQ ID NO: 240 - protein, 7 aa, artificial
  peptide
RRSGGTP

SEQ ID NO: 241 - protein, 7 aa, artificial
  peptide
PPSAAAH

SEQ ID NO: 242 - protein, 7 aa, artificial
  peptide
SAHRSDS

SEQ ID NO: 243 - protein, 7 aa, artificial
  peptide
GAHRSDT

SEQ ID NO: 244 - protein, 7 aa, artificial
  peptide
AQYGAAV

SEQ ID NO: 245 - protein, 7 aa, artificial
  peptide
GAHRSDP

SEQ ID NO: 246 - protein, 7 aa, artificial
  peptide
KGGRSAV

SEQ ID NO: 247 - protein, 7 aa, artificial
  peptide
PPADSRV

SEQ ID NO: 248 - protein, 7 aa, artificial
  peptide
GANRSDS

SEQ ID NO: 249 - protein, 7 aa, artificial
  peptide
RDRATLP

SEQ ID NO: 250 - protein, 7 aa, artificial
  peptide
SISATAR

SEQ ID NO: 251 - protein, 7 aa, artificial
  peptide
TANPGLR

SEQ ID NO: 252 - protein, 7 aa, artificial
  peptide
RGGALGG

SEQ ID NO: 253 - protein, 7 aa, artificial
  peptide
PPDAYRM

SEQ ID NO: 254 - protein, 7 aa, artificial
  peptide
SRASPVA

SEQ ID NO: 255 - protein, 7 aa, artificial
  peptide
RDRATFP

SEQ ID NO: 256 - protein, 7 aa, artificial
  peptide
RDRRDSP

SEQ ID NO: 257 - protein, 7 aa, artificial
  peptide
SGPGGTP

SEQ ID NO: 258 - protein, 7 aa, artificial
  peptide
RRPGGTP

SEQ ID NO: 259 - protein, 7 aa, artificial
  peptide
MRPGNSI

SEQ ID NO: 260 - protein, 7 aa, artificial
  peptide
AGRSPVS

SEQ ID NO: 261 - protein, 7 aa, artificial
  peptide
AARSYSS

SEQ ID NO: 262 - protein, 7 aa, artificial
  peptide
QTNPVRH

SEQ ID NO: 263 - protein, 7 aa, artificial
  peptide
EGGALGG

SEQ ID NO: 264 - protein, 7 aa, artificial
  peptide
LAARDAR

SEQ ID NO: 265 - protein, 7 aa, artificial
  peptide
MPSGTTR

SEQ ID NO: 266 - protein, 7 aa, artificial
  peptide
GAHRSGS

SEQ ID NO: 267 - protein, 7 aa, artificial
  peptide
GTHRSDS

SEQ ID NO: 268 - protein, 7 aa, artificial
  peptide
EGPGDSP

SEQ ID NO: 269 - protein, 7 aa, artificial
  peptide
RDRASSP

SEQ ID NO: 270 - protein, 7 aa, artificial
  peptide
EGPGTSP

SEQ ID NO: 271 - protein, 7 aa, artificial
  peptide
KGGATSP

SEQ ID NO: 272 - protein, 7 aa, artificial
  peptide
EDRATSP

SEQ ID NO: 273 - protein, 7 aa, artificial
  peptide
SRGAPTT

SEQ ID NO: 274 - protein, 7 aa, artificial
  peptide
DSRLKQS

SEQ ID NO: 275 - protein, 7 aa, artificial
  peptide
ANTVLRP

SEQ ID NO: 276 - protein, 12 aa, artificial
  peptide
AAAKGGALGGAA

SEQ ID NO: 277 - protein, 12 aa, artificial
  peptide
AAAASTGPHTAA

SEQ ID NO: 278 - protein, 12 aa, artificial
  peptide
ASATSPSPSRAA

SEQ ID NO: 279 - protein, 12 aa, artificial
  peptide
ASANNPGAPRAA

SEQ ID NO: 280 - protein, 12 aa, artificial
  peptide
ASAGVSPERRAA

SEQ ID NO: 281 - protein, 12 aa, artificial
  peptide
ASAWHAPTHGAA

SEQ ID NO: 282 - protein, 12 aa, artificial
  peptide
ASANAQPPSRAA

SEQ ID NO: 283 - protein, 12 aa, artificial
  peptide
ASAHAAGLPKAA

SEQ ID NO: 284 - protein, 12 aa, artificial
  peptide
AAAPANQSVRAA

SEQ ID NO: 285 - protein, 12 aa, artificial
  peptide
AAAGSNATASAA

SEQ ID NO: 286 - protein, 12 aa, artificial
  peptide
AAAGAHRSDSAA

SEQ ID NO: 287 - protein, 12 aa, artificial
  peptide
AAAANYQGPAAA

SEQ ID NO: 288 - protein, 12 aa, artificial
  peptide
AAANHTAATWAA

SEQ ID NO: 289 - protein, 12 aa, artificial
  peptide
AAATNHASAAAA

SEQ ID NO: 290 - protein, 12 aa, artificial
  peptide
AAANYPGAHSAA

SEQ ID NO: 291 - protein, 12 aa, artificial
  peptide
AAAIGTNSPVAA

SEQ ID NO: 292 - protein, 12 aa, artificial
  peptide
AAAAQYSAAVAA

SEQ ID NO: 293 - protein, 12 aa, artificial
  peptide
AAASYGQSAGAA

SEQ ID NO: 294 - protein, 12 aa, artificial
  peptide
AAAAHGGSIPAA

SEQ ID NO: 295 - protein, 12 aa, artificial
  peptide
AAAYQSGSLPAA

SEQ ID NO: 296 - protein, 12 aa, artificial
  peptide
AAADSQHTQHAA

SEQ ID NO: 297 - protein, 12 aa, artificial
  peptide
AAAGSDRVSTAA

SEQ ID NO: 298 - protein, 12 aa, artificial
  peptide
AAATSNRAATAA

SEQ ID NO: 299 - protein, 12 aa, artificial
  peptide
AAAGGTTLAHAA

SEQ ID NO: 300 - protein, 12 aa, artificial
  peptide
AAASSPNHVAAA

SEQ ID NO: 301 - protein, 12 aa, artificial
  peptide
AAAGNARPPDAA

SEQ ID NO: 302 - protein, 12 aa, artificial
  peptide
AAAPGQTRDSAA

SEQ ID NO: 303 - protein, 12 aa, artificial
  peptide
AAAENATLRTAA

SEQ ID NO: 304 - protein, 12 aa, artificial
  peptide
AAAHTRTSQEAA

SEQ ID NO: 305 - protein, 12 aa, artificial
  peptide
AAAPVGSPSRAA

SEQ ID NO: 306 - protein, 12 aa, artificial
  peptide
AAASSGTTRDAA

SEQ ID NO: 307 - protein, 12 aa, artificial
  peptide
AAADDGRPSRAA

SEQ ID NO: 308 - protein, 12 aa, artificial
  peptide
AAAAAYGAQSAA

SEQ ID NO: 309 - protein, 12 aa, artificial
  peptide
AAAREGSPGRAA

SEQ ID NO: 310 - protein, 12 aa, artificial
  peptide
AAANKTGASPAA

SEQ ID NO: 311 - protein, 12 aa, artificial
  peptide
AAAKASGQTVAA

SEQ ID NO: 312 - protein, 12 aa, artificial
  peptide
AAARDTTTGAAA

SEQ ID NO: 313 - protein, 12 aa, artificial
  peptide
AAAAYGPNAGAA

SEQ ID NO: 314 - protein, 12 aa, artificial
  peptide
AAADSDGPRPAA

SEQ ID NO: 315 - protein, 12 aa, artificial
  peptide
AAAGSSATASAA

SEQ ID NO: 316 - protein, 12 aa, artificial
  peptide
AAATSAGSPRAA

SEQ ID NO: 317 - protein, 12 aa, artificial
  peptide
AAAGPAHTSSAA

SEQ ID NO: 318 - protein, 12 aa, artificial
  peptide
AAANAAGTPRAA

SEQ ID NO: 319 - protein, 12 aa, artificial
  peptide
AAANRTALGSAA

SEQ ID NO: 320 - protein, 12 aa, artificial
  peptide
AAAKSHSASPAA

SEQ ID NO: 321 - protein, 12 aa, artificial
  peptide
AAARGHTAPIAA

SEQ ID NO: 322 - protein, 12 aa, artificial
  peptide
AAAPTSGAPKAA

SEQ ID NO: 323 - protein, 12 aa, artificial
  peptide
AAAGNRVQSTAA

SEQ ID NO: 324 - protein, 12 aa, artificial
  peptide
AAANTSLTARAA

SEQ ID NO: 325 - protein, 12 aa, artificial
  peptide
AAARDRATSPAA

SEQ ID NO: 326 - protein, 12 aa, artificial
  peptide
AAAGAERTYSAA

SEQ ID NO: 327 - protein, 12 aa, artificial
  peptide
AAAARGSVSPAA

SEQ ID NO: 328 - protein, 12 aa, artificial
  peptide
AAATGRSSGDAA

SEQ ID NO: 329 - protein, 12 aa, artificial
  peptide
AAAHPARPNAAA

SEQ ID NO: 330 - protein, 12 aa, artificial
  peptide
AAASDHLHPRAA

SEQ ID NO: 331 - protein, 12 aa, artificial
  peptide
AAATVLSSQRAA

SEQ ID NO: 332 - protein, 12 aa, artificial
  peptide
AAAVSSSSRPAA

SEQ ID NO: 333 - protein, 12 aa, artificial
  peptide
AAATGSSKGVAA

SEQ ID NO: 334 - protein, 12 aa, artificial
  peptide
AAASPGGPARAA

SEQ ID NO: 335 - protein, 12 aa, artificial
  peptide
AAAGRSEQIRAA

SEQ ID NO: 336 - protein, 12 aa, artificial
  peptide
AAAVTARPEGAA

SEQ ID NO: 337 - protein, 12 aa, artificial
  peptide
AAARDPSPGRAA

SEQ ID NO: 338 - protein, 12 aa, artificial
  peptide
AAAAASGVARAA

SEQ ID NO: 339 - protein, 12 aa, artificial
  peptide
AAATSARSVPAA

SEQ ID NO: 340 - protein, 12 aa, artificial
  peptide
AAAARGGSIPAA

SEQ ID NO: 341 - protein, 12 aa, artificial
  peptide
AAARAAGQVAAA

SEQ ID NO: 342 - protein, 12 aa, artificial
  peptide
AAATSQQRIPAA

SEQ ID NO: 343 - protein, 12 aa, artificial
  peptide
AAANSARPNSAA

SEQ ID NO: 344 - protein, 12 aa, artificial
  peptide
AAAPVGSPGRAA

SEQ ID NO: 345 - protein, 12 aa, artificial
  peptide
AAATQSPPGRAA

SEQ ID NO: 346 - protein, 12 aa, artificial
  peptide
AAAGRRDSPVAA

SEQ ID NO: 347 - protein, 12 aa, artificial
  peptide
AAANGSPVGRAA

SEQ ID NO: 348 - protein, 12 aa, artificial
  peptide
AAAKVSGHQAAA

SEQ ID NO: 349 - protein, 12 aa, artificial
  peptide
AAAARSQVHAAA

SEQ ID NO: 350 - protein, 12 aa, artificial
  peptide
AAAARTTHSPAA

SEQ ID NO: 351 - protein, 12 aa, artificial
  peptide
AAANLSRASPAA

SEQ ID NO: 352 - protein, 12 aa, artificial
  peptide
AAANTPQSARAA

SEQ ID NO: 353 - protein, 12 aa, artificial
  peptide
AAAGRPGSGYAA

SEQ ID NO: 354 - protein, 12 aa, artificial
  peptide
AAAVPSAPQRAA

SEQ ID NO: 355 - protein, 12 aa, artificial
  peptide
AAAAPANLPRAA

SEQ ID NO: 356 - protein, 12 aa, artificial
  peptide
AAALGRPAGPAA

SEQ ID NO: 357 - protein, 12 aa, artificial
  peptide
AAAVLGTKGAAA

SEQ ID NO: 358 - protein, 12 aa, artificial
  peptide
AAAGRPEQIRAA

SEQ ID NO: 359 - protein, 12 aa, artificial
  peptide
AAAGLAGVVRAA

SEQ ID NO: 360 - protein, 12 aa, artificial
  peptide
AAAAHRSTGTAA

SEQ ID NO: 361 - protein, 12 aa, artificial
  peptide
AAAGTSPANRAA

SEQ ID NO: 362 - protein, 12 aa, artificial
  peptide
AAANRPNAASAA

SEQ ID NO: 363 - protein, 12 aa, artificial
  peptide
AAAARSGSGAAA

SEQ ID NO: 364 - protein, 12 aa, artificial
  peptide
AAARHSSTPVAA

SEQ ID NO: 365 - protein, 12 aa, artificial
  peptide
AAANSPLGTRAA

SEQ ID NO: 366 - protein, 12 aa, artificial
  peptide
AAANTPSPARAA

SEQ ID NO: 367 - protein, 12 aa, artificial
  peptide
AAANSPAAGRAA

SEQ ID NO: 368 - protein, 12 aa, artificial
  peptide
AAASNRTSSVAA

SEQ ID NO: 369 - protein, 12 aa, artificial
  peptide
AAASVRGGTPAA

SEQ ID NO: 370 - protein, 12 aa, artificial
  peptide
AAASPADSRVAA

SEQ ID NO: 371 - protein, 12 aa, artificial
  peptide
AAASTRGQGHAA

SEQ ID NO: 372 - protein, 12 aa, artificial
  peptide
AAAVDSGLSGAA

SEQ ID NO: 373 - protein, 12 aa, artificial
  peptide
AAAGDGSFSGAA

SEQ ID NO: 374 - protein, 12 aa, artificial
  peptide
AAAVSGPPHTAA

SEQ ID NO: 375 - protein, 12 aa, artificial
  peptide
AAARHGPQHPAA

SEQ ID NO: 376 - protein, 12 aa, artificial
  peptide
AAARDRAASPAA

SEQ ID NO: 377 - protein, 12 aa, artificial
  peptide
AAAGDRATSPAA

SEQ ID NO: 378 - protein, 12 aa, artificial
  peptide
AAARGRATSPAA

SEQ ID NO: 379 - protein, 12 aa, artificial
  peptide
AAARDRVTSPAA

SEQ ID NO: 380 - protein, 12 aa, artificial
  peptide
AAAPVLNPPPAA

SEQ ID NO: 381 - protein, 12 aa, artificial
  peptide
AAASFQRASPAA

SEQ ID NO: 382 - protein, 12 aa, artificial
  peptide
AAARDRATTPAA

SEQ ID NO: 383 - protein, 12 aa, artificial
  peptide
AAAQSRLSTQAA

SEQ ID NO: 384 - protein, 12 aa, artificial
  peptide
AAASSGGGTPAA

SEQ ID NO: 385 - protein, 12 aa, artificial
  peptide
AAAKGEALGGAA

SEQ ID NO: 386 - protein, 12 aa, artificial
  peptide
AAASVRGAPPAA

SEQ ID NO: 387 - protein, 12 aa, artificial
  peptide
AAADHDSPSVAA

SEQ ID NO: 388 - protein, 12 aa, artificial
  peptide
AAARDQATSPAA

SEQ ID NO: 389 - protein, 12 aa, artificial
  peptide
AAARDRATSSAA

SEQ ID NO: 390 - protein, 12 aa, artificial
  peptide
AAAGKPPRTEAA

SEQ ID NO: 391 - protein, 12 aa, artificial
  peptide
AAAAHRGLQVAA

SEQ ID NO: 392 - protein, 12 aa, artificial
  peptide
AAAVASLAARAA

SEQ ID NO: 393 - protein, 12 aa, artificial
  peptide
AAARDRTTSPAA

SEQ ID NO: 394 - protein, 12 aa, artificial
  peptide
AAARDWATSPAA

SEQ ID NO: 395 - protein, 12 aa, artificial
  peptide
AAARNRATSPAA

SEQ ID NO: 396 - protein, 12 aa, artificial
  peptide
AAARDRATSLAA

SEQ ID NO: 397 - protein, 12 aa, artificial
  peptide
AAAKDRATSPAA

SEQ ID NO: 398 - protein, 12 aa, artificial
  peptide
AAAARYSAAVAA

SEQ ID NO: 399 - protein, 12 aa, artificial
  peptide
AAAGAHRSVSAA

SEQ ID NO: 400 - protein, 12 aa, artificial
  peptide
AAAWDRATSPAA

SEQ ID NO: 401 - protein, 12 aa, artificial
  peptide
AAAERPYQAGAA

SEQ ID NO: 402 - protein, 12 aa, artificial
  peptide
AAANAPGNLRAA

SEQ ID NO: 403 - protein, 12 aa, artificial
  peptide
AAANNPLPQRAA

SEQ ID NO: 404 - protein, 12 aa, artificial
  peptide
AAAARPTVPYAA

SEQ ID NO: 405 - protein, 12 aa, artificial
  peptide
AAARSGAAVAAA

SEQ ID NO: 406 - protein, 12 aa, artificial
  peptide
AAANTPNPLRAA

SEQ ID NO: 407 - protein, 12 aa, artificial
  peptide
AAAGGTTLGRAA

SEQ ID NO: 408 - protein, 12 aa, artificial
  peptide
AAAASTGLPRAA

SEQ ID NO: 409 - protein, 12 aa, artificial
  peptide
AAAATGVIQRAA

SEQ ID NO: 410 - protein, 12 aa, artificial
  peptide
AAANSAALGRAA

SEQ ID NO: 411 - protein, 12 aa, artificial
  peptide
AAAGEPQPRFAA

SEQ ID NO: 412 - protein, 12 aa, artificial
  peptide
AAAGHSARSIAA

SEQ ID NO: 413 - protein, 12 aa, artificial
  peptide
AAAVSSSTPRAA

SEQ ID NO: 414 - protein, 12 aa, artificial
  peptide
AAANQPNPVRAA

SEQ ID NO: 415 - protein, 12 aa, artificial
  peptide
AAAPSGGPPRAA

SEQ ID NO: 416 - protein, 12 aa, artificial
  peptide
AAAARDQLPRAA

SEQ ID NO: 417 - protein, 12 aa, artificial
  peptide
AAARGSHPNIAA

SEQ ID NO: 418 - protein, 12 aa, artificial
  peptide
AAANRASNTSAA

SEQ ID NO: 419 - protein, 12 aa, artificial
  peptide
AAAVNAVPGRAA

SEQ ID NO: 420 - protein, 12 aa, artificial
  peptide
AAAPANTPGRAA

SEQ ID NO: 421 - protein, 12 aa, artificial
  peptide
AAAISSSTARAA

SEQ ID NO: 422 - protein, 12 aa, artificial
  peptide
AAAARAGRDLAA

SEQ ID NO: 423 - protein, 12 aa, artificial
  peptide
AAARGGEVPRAA

SEQ ID NO: 424 - protein, 12 aa, artificial
  peptide
AAAVNLVPSRAA

SEQ ID NO: 425 - protein, 12 aa, artificial
  peptide
AAALARNQSGAA

SEQ ID NO: 426 - protein, 12 aa, artificial
  peptide
AAASSSGSPRAA

SEQ ID NO: 427 - protein, 12 aa, artificial
  peptide
AAASPGINLRAA

SEQ ID NO: 428 - protein, 12 aa, artificial
  peptide
AAAAVAGSPRAA

SEQ ID NO: 429 - protein, 12 aa, artificial
  peptide
AAARSPDSARAA

SEQ ID NO: 430 - protein, 12 aa, artificial
  peptide
AAANQPVPNRAA

SEQ ID NO: 431 - protein, 12 aa, artificial
  peptide
AAAPSSPVARAA

SEQ ID NO: 432 - protein, 12 aa, artificial
  peptide
AAAKDAQLGRAA

SEQ ID NO: 433 - protein, 12 aa, artificial
  peptide
AAAVTSSSGKAA

SEQ ID NO: 434 - protein, 12 aa, artificial
  peptide
AAAAANMTARAA

SEQ ID NO: 435 - protein, 12 aa, artificial
  peptide
AAASSINPPRAA

SEQ ID NO: 436 - protein, 12 aa, artificial
  peptide
AAAAYAGTPKAA

SEQ ID NO: 437 - protein, 12 aa, artificial
  peptide
AAARGSSPVRAA

SEQ ID NO: 438 - protein, 12 aa, artificial
  peptide
AAAGGGLTPRAA

SEQ ID NO: 439 - protein, 12 aa, artificial
  peptide
AAAGASSVSRAA

SEQ ID NO: 440 - protein, 12 aa, artificial
  peptide
AAAVHLSPGRAA

SEQ ID NO: 441 - protein, 12 aa, artificial
  peptide
AAANSPNVARAA

SEQ ID NO: 442 - protein, 12 aa, artificial
  peptide
AAAGGRDQLRAA

SEQ ID NO: 443 - protein, 12 aa, artificial
  peptide
AAAAAQGMPKAA

SEQ ID NO: 444 - protein, 12 aa, artificial
  peptide
AAARRDLDVRAA

SEQ ID NO: 445 - protein, 12 aa, artificial
  peptide
AAAVTSTSARAA

SEQ ID NO: 446 - protein, 12 aa, artificial
  peptide
AAARHGSPGSAA

SEQ ID NO: 447 - protein, 12 aa, artificial
  peptide
AAANSPSGPRAA

SEQ ID NO: 448 - protein, 12 aa, artificial
  peptide
AAARDGITSRAA

SEQ ID NO: 449 - protein, 12 aa, artificial
  peptide
AAASNAPSARAA

SEQ ID NO: 450 - protein, 12 aa, artificial
  peptide
AAAVMNGQLRAA

SEQ ID NO: 451 - protein, 12 aa, artificial
  peptide
AAAYSAGSPKAA

SEQ ID NO: 452 - protein, 12 aa, artificial
  peptide
AAASDNHTARAA

SEQ ID NO: 453 - protein, 12 aa, artificial
  peptide
AAARDRAGPPAA

SEQ ID NO: 454 - protein, 12 aa, artificial
  peptide
AAARVRATSPAA

SEQ ID NO: 455 - protein, 12 aa, artificial
  peptide
AAASTRGHGAAA

SEQ ID NO: 456 - protein, 12 aa, artificial
  peptide
AAASTRGRGAAA

SEQ ID NO: 457 - protein, 12 aa, artificial
  peptide
AAANDVRAPPAA

SEQ ID NO: 458 - protein, 12 aa, artificial
  peptide
AAASNGGSPVAA

SEQ ID NO: 459 - protein, 12 aa, artificial
  peptide
AAAMHASPSGAA

SEQ ID NO: 460 - protein, 12 aa, artificial
  peptide
AAAAASHPNQAA

SEQ ID NO: 461 - protein, 12 aa, artificial
  peptide
AAADTGSHALAA

SEQ ID NO: 462 - protein, 12 aa, artificial
  peptide
AAASPSDRFSAA

SEQ ID NO: 463 - protein, 12 aa, artificial
  peptide
AAAGPNATASAA

SEQ ID NO: 464 - protein, 12 aa, artificial
  peptide
AAAGGPGYTAAA

SEQ ID NO: 465 - protein, 12 aa, artificial
  peptide
AAAGSDATASAA

SEQ ID NO: 466 - protein, 12 aa, artificial
  peptide
AAAAQYSAAAAA

SEQ ID NO: 467 - protein, 12 aa, artificial
  peptide
AAAVPTLRPAAA

SEQ ID NO: 468 - protein, 12 aa, artificial
  peptide
AAAAGTGIPVAA

SEQ ID NO: 469 - protein, 12 aa, artificial
  peptide
AAAGSNATAGAA

SEQ ID NO: 470 - protein, 12 aa, artificial
  peptide
AAAGPANSSHAA

SEQ ID NO: 471 - protein, 12 aa, artificial
  peptide
AAAAARSTDLAA

SEQ ID NO: 472 - protein, 12 aa, artificial
  peptide
AAASHGGLSEAA

SEQ ID NO: 473 - protein, 12 aa, artificial
  peptide
AAAGQAMSGGAA

SEQ ID NO: 474 - protein, 12 aa, artificial
  peptide
AAAGTNATASAA

SEQ ID NO: 475 - protein, 12 aa, artificial
  peptide
AAAFTAANNQAA

SEQ ID NO: 476 - protein, 12 aa, artificial
  peptide
AAASSNATASAA

SEQ ID NO: 477 - protein, 12 aa, artificial
  peptide
AAAGSNTTASAA

SEQ ID NO: 478 - protein, 12 aa, artificial
  peptide
AAAVSPNHAAAA

SEQ ID NO: 479 - protein, 12 aa, artificial
  peptide
AAAGSNAAASAA

SEQ ID NO: 480 - protein, 12 aa, artificial
  peptide
AAANVRPHPAAA

SEQ ID NO: 481 - protein, 12 aa, artificial
  peptide
AAANSERSVQAA

SEQ ID NO: 482 - protein, 12 aa, artificial
  peptdie
AAATVGRDSVAA

SEQ ID NO: 483 - protein, 12 aa, artificial
  peptide
AAAPSDSTDRAA

SEQ ID NO: 484 - protein, 12 aa, artificial
  peptide
AAAPANTPPRAA

SEQ ID NO: 485 - protein, 12 aa, artificial
  peptide
AAAGANRPGLAA

SEQ ID NO: 486 - protein, 12 aa, artificial
  peptide
AAATNHAGAAAA

SEQ ID NO: 487 - protein, 12 aa, artificial
  peptide
AAAASTGPRTAA

SEQ ID NO: 488 - protein, 12 aa, artificial
  peptde
AAASSYAPSSAA

SEQ ID NO: 489 - protein, 12 aa, artificial
  peptide
AAAAQPPQPPAA

SEQ ID NO: 490 - protein, 12 aa, artificial
  peptide
AAAAPTGPHTAA

SEQ ID NO: 491 - protein, 12 aa, artificial
  peptide
AAAAHLSGTSAA

SEQ ID NO: 492 - protein, 12 aa, artificial
  peptde
AAATRSGDSSAA

SEQ ID NO: 493 - protein, 12 aa, artificial
  peptide
AAARTGRTSPAA

SEQ ID NO: 494 - protein, 12 aa, artificial
  peptide
AAARTGRDSPAA

SEQ ID NO: 495 - protein, 12 aa, artificial
  peptide
AAADDFQRARAA

SEQ ID NO: 496 - protein, 12 aa, artificial
  peptide
AAARDRADSPAA

SEQ ID NO: 497 - protein, 12 aa, artificial
  peptide
AAARDRAISPAA

SEQ ID NO: 498 - protein, 12 aa, artificial
  peptide
AAARDRRTSPAA

SEQ ID NO: 499 - protein, 12 aa, artificial
  peptide
AAAGARRSDSAA

SEQ ID NO: 500 - protein, 12 aa, artificial
  peptide
AAARTGGTSPAA

SEQ ID NO: 501 - protein, 12 aa, artificial
  peptide
AAARTGATSPAA

SEQ ID NO: 502 - protein, 12 aa, artificial
  peptide
AAAGAHRPDSAA

SEQ ID NO: 503 - protein, 12 aa, artificial
  peptide
AAARRSGGTPAA

SEQ ID NO: 504 - protein, 12 aa, artificial
  peptide
AAAPPSAAAHAA

SEQ ID NO: 505 - protein, 12 aa, artificial
  peptide
AAASAHRSDSAA

SEQ ID NO: 506 - protein, 12 aa, artificial
  peptide
AAAGAHRSDTAA

SEQ ID NO: 507 - protein, 12 aa, artificial
  peptide
AAAAQYGAAVAA

SEQ ID NO: 508 - protein, 12 aa, artificial
  peptide
AAAGAHRSDPAA

SEQ ID NO: 509 - protein, 12 aa, artificial
  peptide
AAAKGGRSAVAA

SEQ ID NO: 510 - protein, 12 aa, artificial
  peptide
AAAPPADSRVAA

SEQ ID NO: 511 - protein, 12 aa, artificial
  peptide
AAAGANRSDSAA

SEQ ID NO: 512 - protein, 12 aa, artificial
  peptide
AAARDRATLPAA

SEQ ID NO: 513 - protein, 12 aa, artificial
  peptide
AAASISATARAA

SEQ ID NO: 514 - protein, 12 aa, artificial
  peptide
AAATANPGLRAA

SEQ ID NO: 515 - protein, 12 aa, artificial
  peptide
AAARGGALGGAA

SEQ ID NO: 516 - protein, 12 aa, artificial
  peptide
AAAPPDAYRMAA

SEQ ID NO: 517 - protein, 12 aa, artificial
  peptide
AAASRASPVAAA

SEQ ID NO: 518 - protein, 12 aa, artificial
  peptide
AAARDRATFPAA

SEQ ID NO: 519 - protein, 12 aa, artificial
  peptide
AAARDRRDSPAA

SEQ ID NO: 520 - protein, 12 aa, artificial
  peptide
AAASGPGGTPAA

SEQ ID NO: 521 - protein, 12 aa, artificial
  peptide
AAARRPGGTPAA

SEQ ID NO: 522 - protein, 12 aa, artificial
  peptide
AAAMRPGNSIAA

SEQ ID NO: 523 - protein, 12 aa, artificial
  peptide
AAAAGRSPVSAA

SEQ ID NO: 524 - protein, 12 aa, artificial
  peptide
AAAAARSYSSAA

SEQ ID NO: 525 - protein, 12 aa, artificial
  peptide
AAAQTNPVRHAA

SEQ ID NO: 526 - protein, 12 aa, artificial
  peptide
AAAEGGALGGAA

SEQ ID NO: 527 - protein, 12 aa, artificial
  peptide
AAALAARDARAA

SEQ ID NO: 528 - protein, 12 aa, artificial
  peptide
AAAMPSGTTRAA

SEQ ID NO: 529 - protein, 12 aa, artificial
  peptide
AAAGAHRSGSAA

SEQ ID NO: 530 - protein, 12 aa, artificial
  peptide
AAAGTHRSDSAA

SEQ ID NO: 531 - protein, 12 aa, artificial
  peptide
AAAEGPGDSPAA

SEQ ID NO: 532 - protein, 12 aa, artificial
  peptide
AAARDRASSPAA

SEQ ID NO: 533 - protein, 12 aa, artificial
  peptide
AAAEGPGTSPAA

SEQ ID NO: 534 - protein, 12 aa, artificial
  peptide
AAAKGGATSPAA

SEQ ID NO: 535 - protein, 12 aa, artificial
  peptide
AAAEDRATSPAA

SEQ ID NO: 536 - protein, 12 aa, artificial
  peptide
AAASRGAPTTAA

SEQ ID NO: 537 - protein, 12 aa, artificial
  peptide
AAADSRLKQSAA

SEQ ID NO: 538 - protein, 12 aa, artificial
  peptide
AAAANTVLRPAA

(19) United States
(12) Patent Application Publication
(10) Pub. No.: US 2022/0184227 A1
(43) Pub. Date: Jun. 16, 2022

(54) MUTATED ADENO-ASSOCIATED VIRUS CAPSID PROTEINS, AAV PARTICLE COMPRISING THE SAME AND LIVER DIRECTED AAV VECTOR GENE THERAPY

(71) Applicant: Fraunhofer Gesellschaft zur Föndrung der angeewandten Forschung e.V., München (DE)

(72) Inventors: Hildegard BÜNING, Hannover (DE); Nadja MEUMANN, Hannover (DE); Maria GONZALEZ-CARMONA, München (DE); Christian P. STRASSBURG, München (DE); Annabelle VOGT, München (DE); Albrecht PIIPER, München (DE); Joachim SCHWÄBLE, München (DE); Karin HUBER, München (DE); Erhard SEIFRIED, München (DE)

(21) Appl. No.: 17/599,235

(22) PCT Filed: Mar. 30, 2020
(86) PCT No.: PCT/EP2020/058895

§ 371 Date: Sep. 28, 2021

(30) Foreign Application Priority Data
Mar. 28, 2019 (EP) 19165681.8

(51) Int. Cl. A61K 48/00; C07K 14/005; C12N 15/86

(52) U.S. Cl. CPC A61K 48/005; C07K 14/005; C12N 2750/14145; C07K 2319/33; C12N 2750/14122; C12N 15/86

(57) ABSTRACT

The present invention relates in a first aspect to a mutated adeno-associated virus (AAV) capsid protein or fragment thereof having an insert composed of an oligopeptide optionally having further amino acids representing linker sequences flanking both sites of said oligopeptide. These mutated AAV capsid proteins are more efficient in targeting hepatic tissue, hepatocytes, hepatic cells and cell lines or hepatocellular carcinoma (HCC) with partially higher specificity. Further, a mutated AAV particle comprising the mutated AAV capsid protein according to the present invention is provided. In addition, a nucleic acid encoding the mutated AAV capsid protein according to the present invention is identified together with a corresponding nucleic acid vector, in particular, a plasmid. In addition, a host cell containing the nucleic acid vector or the nucleic acid molecule according to the present invention. Further, a use of the AAV particle or the nucleic acid or the nucleic acid vector according to the present invention in the manufacture of an AAV particle, or in the manufacture of a medicament for gene therapy is described. Moreover, the described protein, particle molecules as well as nucleic acid vectors for use in targeting hepatocytes and/or HCC are de-scribed. In particular, said components for use in treating diseases involving hepatocytes or for treating HCC are disclosed, in particular, for use in gene therapy, e.g. for use in a transfer of a gene of interest into hepatocytes, heptatic tissue or HCC.

The present invention relates in a first aspect to a mutated adeno-associated virus (AAV) capsid protein or fragment thereof having an insert composed of an oligopeptide optionally having further amino acids representing linker sequences at both sites of said oligopeptide. These mutated AAV capsid proteins are more efficient in transferring genetic material into hepatic tissue, hepatocytes, hepatic cells and cell lines or hepatocellular carcinoma (HCC) partially with improved tropism for these cell types. Further, a mutated AAV particle (uniform or as hybrid) comprising the mutated AAV capsid protein according to the present invention is provided. In addition, a nucleic acid encoding the mutated AAV capsid protein according to the present invention is identified together with a corresponding nucleic acid vector, in particular, a plasmid. In addition, a host cell containing the nucleic acid vector or the nucleic acid molecule according to the present invention. Further, a use of the AAV particle or the nucleic acid or the nucleic acid vector according to the present invention in the manufacture of a medicament for gene therapy. Moreover, the described protein, particle molecules as well as nucleic acid vectors for use in targeting hepatocytes and/or HCC are described. In particular, said components for use in treating diseases involving hepatocytes or for treating HCC are disclosed, in particular, for use in gene therapy, e.g. for use in a transfer of a gene of interest into hepatocytes, hepatic tissue or HCC.

### BACKGROUND OF THE INVENTION

The liver represents an essential organ with complex metabolic, immunological and hemostatic functions. The liver is affected by a broad range of monogenic disorders and chronic metabolic conditions, see Marcellin, P. and Kutala, B. K., Liver International, 2018, 38(S1), 2-6. Consequently, liver has become a key target for the development of gene therapy strategies in particular with AAV vectors as summarized in Baruteau, J., Journal of Inherited Metabolic Disease, 2017, 497-517.

As reviewed recently, promising results, for example, have been obtained for patients suffering from hemophilia, see Pierce, G. F. and Iorio, A., Hamophilia, 1018, 24(Suppl 6), 60-67. However, still high vector doses are required to compensate for the low efficacy of AAV vectors in human patients. While efficacy is sufficient to obtain the moderate physiological levels of blood clotting factors required to transform severe hemophilic phenotypes into mild forms, other liver diseases are in urgent need for more effective AAV vectors. In addition, patients establish neutralizing capsid antibodies upon the first AAV vector dose, as described by Fitzpatrick et al., Molecular Therapy: Methods & Clinical Development. Elsevier Ltd., 9 (June), 119-129. This impairs re-administration of AAV vectors with the same or cross-reacting AAV capsids for improving transgene expression levels. Therefore, AAV vectors with novel capsid properties for efficient liver-directed gene transfer are of utmost importance for improving current and future gene therapeutic approaches.

Beside the above-mentioned diseases, liver is prone to develop cancer, in particular hepatocellular carcinoma (HCC), or to being the “home” for metastasis originating from tumors other than HCC. With regard to HCC, it is reported to be the third most common cause of cancer-related deaths worldwide. It is a severe cancer disease with poor prognosis at advanced HCC stages. So far potentially curative treatments are limited to surgical procedures like tumor resection, orthotopic liver transplantation, or percutaneous radio frequency ablation, see Waghray, A. et al., WJH, 2015, 7(8), 1020-1029. The majority of patients are diagnosed at advanced stages of HCC for which current therapeutic options are very poor. Therefore, there is a strong need for novel therapeutic approaches including AAV vector-based cancer gene therapy. AAV vectors may be used to deliver suicide genes, tumor-suppressor genes or immunotherapeutic transgenes to the tumor site, e.g. as discussed in Dhungel, B. et al., 2017, 7544. To provide efficient and safe delivery of transgene for cancer gene therapy, engineering of novel AAV vectors is required which are directed to the HCC target tissue and are capable of efficient therapeutic transgene expression.

AAV vectors are based on the adeno-associated virus (AAV), a non-pathogenic member of the parvoviridae and the genus Dependoparvovirus. AAV are composed of a single-stranded DNA genome of about 4.7 kB packaged into a non-enveloped icosahedral protein capsid. It is a small (25 nm in diameter) virus, consisting of 60 monomers forming the icosahedral capsid. These monomers are composed of the viral capsid proteins (VP) VP1, VP2, and VP3 in a 1:1:10 ratio. The 4.5 kB single stranded coding DNA genome (plus inverted terminal repeats (ITRs)) is bearing two genes (rep and cap) encoding for non-structural (Rep proteins and assembly activating protein) and structural proteins (VP1, VP2, VP3). The AAV genome is flanked by ITRs, which serve as packaging and replication signals. AAV infection biology is mainly based on the interaction of the AAV capsid with the host cell. Specifically, the capsid is interacting with cell surface receptors of the host cell mediating cell entry and intracellular trafficking as well as processing of the virion. AAV has not been associated with any disease; rather, discussion arises about being tumor protective. It is unique in being dependent on the presence of a helper virus like adenovirus for progeny production. Further, AAV is able to transduce mitotic and postmitotic tissues and shows a broad tissue tropism. In fact, for in vivo application a major drawback of AAV vectors, in particular, AAV serotype 2-based vectors, is their broad tropism. Since AAV vectors are able to transduce a wide range of cell types, transduction efficacy of a distinct target organ is lowered, and administration of higher vector doses are required to achieve therapeutic target cell transduction levels.

However, AAV, in particular, AAV2, has gained tremendous popularity as vector for gene therapy, but also as platform for vaccine development and as tool in preclinical research. AAV vectors are considered as gene delivery system of choice for in vivo gene therapy. Advantageous features of AAV include its apathogenicity, high stability, ability to transduce both dividing and non-dividing cells, long-term gene expression in post mitotic or slowly proliferating cells and low immunogenicity. In addition, AAV vectors lack an intrinsic genome integration activity and are therefore defined as non-integrating vector system. This is a main advantage compared to retro-/lentiviral vectors as it significantly reduces the risk of insertional mutagenesis. Further, vectors can be produced to high titer and purity owing to its stability.

To date, 13 different natural human and non-human primate AAV serotypes and over 100 natural variants have been isolated. Most of the serotypes have been vectorized as alternatives to AAV serotype 2, the prototype AAV vector, because they differ in tropism and in epitopes recognized by the immune system. For vectorization viral genomes are replaced by a transgene cassette. Usage of serotypes is simplified by the pseudopackaging technology allowing to package ITR2-flanked AAV vector genomes into non-AAV2 capsids. This enables the production of AAV vectors differing in capsid but delivering the same vector genome. The AAV vector capsid and genome can be modified and optimized for efficient and directed gene delivery in vitro and in vivo.

Pseudotyping (pseudopackaging) represents a strategy to change the tropism of the AAV vector system. However, it does not allow to re-direct the tropism towards a distinct cell type. In addition, its potency of increasing efficiency of AAV vector-mediated cell transduction is limited. Therefore, alternative strategies summarized as capsid-engineering technologies have been developed to tailor the host-AAV interaction including the first step of cell transduction. Consequently, non-permissive cell types became susceptible towards AAV transduction and transduction efficiencies for permissive cells could be improved. In addition, it became possible to redirect viral vector in vivo tropism towards a defined cell surface structure (Büning, H. et al., Current Opinion in Pharmacology, 2015, 24, 94-104).

In genetic capsid engineering approaches which focus on cell surface targeting, peptide ligands are inserted into a suitable position of the capsid. This position needs to be exposed on the capsid surface to influence target cell infection properties. To date, peptide insertions for tropism modification are mainly performed by the following techniques: i) fusion to the N-terminus of VP2 or ii) insertion of peptides at the tip of variable region (VR)-VIII, for AAV2: insertion at amino acid position 587 and insertion at position 588, or insertion at VR-IV, for AAV2: insertion at amino acid position 453 as described in Büning et al., see above; and Büning and Svrivastava, Mol Ther Methods Clin Dev, 2019, 12, 248-265. For example, EP 2 158 211 B1 identifies structure protein insertions having a length of 4 to 13 amino acids, which represent ligands mediating cell targeting accordingly. In addition, approaches have been conducted for substituting specific amino acids present in the capsid proteins.

Since for a lot of target cells or tissues of therapeutic interest suitable peptide ligands that would mediate target cell transduction are unknown, high-throughput screenings of AAV peptide display libraries have been described. For example, libraries have been disclosed in Müller, O. J. et al., Nature Biotechnology, 2003, 21(9), 1040-1046 and Perabo, L. et al., Molecular Therapy, 2003, 8(1), 151-157. The AAV peptide display library established by Perabo et al. is composed of >4 E6 capsid variants. The library has been generated by insertion of a random 7-mer peptide into amino acid position 587 (VP1 numbering) of AAV2 capsid proteins. The random peptide is flanked by linker sequences to help optimal presentation. This library is described for example in EP 1 456 383 B1 and also in EP 2 363 487 A2. Selection of this library on target cells has been used to identify AAV capsid variants with novel and improved transduction properties.

In US 2002/0192823 A1 an AAV capsid variant and methods have been described whereby said AAV capsid variant is derived from a shuffled AAV capsid library based on AAV2, AAV8, and AAV9.

Further strategies include attachment of foreign molecules to the outer surface of the vector that can mediate specific interaction with cellular receptors expressed on the membrane of target cell types. Chemical modifications of amino acids present on the surface or on the exterior of the capsid surface have been discussed. These modifications include binding of biotin for allowing coupling of further components, e.g. via streptavidin. Further, introduction of unnatural moieties have been disclosed. For example, WO 2012/149160 A2 describes the strategy for modification of virus capsid proteins based on introducing unnatural amino acid moieties which eventually allows to introduce further groups.

As noted, a main problem of the AAV vector system is the in vivo tropism as well as the transduction efficiency of specific tissue types, thus, requiring the use of high vector doses.

### SUMMARY OF THE PRESENT INVENTION

Consequently, it is an object of the present invention to provide AAV vectors and capsid proteins overcoming the problems described in the art, namely, the problem of broad tropism and low transduction efficiency.

The present invention relates in a first aspect to a mutated adeno-associated virus (AAV) capsid protein or a fragment thereof wherein an insert is inserted after at least one of the amino acids having the amino acid numbers 139, 161, 261, 381, 447, 453, 459, 534, 570, 573, 584, 585, 586, 587, 588, 589 of SEQ ID No. 2 corresponding to the capsid protein of AAV2 or the homolog capsid protein of the other serotypes of AAV, namely, AAV1, AAV3b to AAV11, and AAV bovine, the insert comprises an oligonucleotide of at least four amino acids and at most thirty amino acids, like consisting of five to twelve amino acids, in particular, seven amino acids, said insert may optionally contain further amino acids representing a linker sequence at both sites of said oligopeptide, said linker sequence having independently from one another a size of 1 to 3 amino acids, if present; wherein the mutated AAV capsid protein or a fragment thereof or its homologs being more efficient in targeting hepatic tissue, hepatocytes, hepatic cells and cell lines or hepatocellular carcinoma (HCC) with higher specificity.

In a second aspect, the present invention relates to a mutated AAV particle comprising the AAV capsid protein or fragment thereof according to the present invention. Further, the present invention relates to a nucleic acid encoding the AAV virus capsid protein or fragment thereof according to the present invention. In addition, a nucleic acid vector, in particular, a plasmid comprising the nucleic acid molecules according to the present invention is disclosed as well as host cells containing said nucleic acid vector or said nucleic acid according to the present invention. Moreover, the use of the AAV particle according to the present invention or the nucleic acid molecule according to the present invention or the nucleic acid vector according to the present invention in the manufacture of an AAV particle, or a manufacture of a medicament for gene therapy. Moreover, the components described herein including the mutated AAV capsid protein or a fragment thereof or a homolog as well as the mutated AAV particle or the nucleic acid molecule or the nucleic acid vector are suitable for use in targeting hepatocytes and/or HCC and/or improving transduction efficiency of AAV vectors in hepatocytes and/or HCC. Further, these components are suitable for use in transfer of a gene of interest into hepatocytes or HCC, in particular, for use in gene therapy.

That is, the present inventors identified new variants of mutated AAV capsid proteins or fragments thereof being more efficient in transducing hepatic tissue, hepatocytes, hepatic cells and cell lines, and HCC, respectively, with higher specificity and/or efficiency.

### BRIEF DESCRIPTION OF THE DRAWINGS

FIG. 1: Mean transgene expression of rAAV vectors in Balb/c mice (A) over the course of 28 days and (B) at day 28. Animals received 4.8E11 particles of indicated AAV vectors by tail vein injection. After 7, 15, and 28 days mice were analyzed for vector transduction. Specifically, animals received 1.5 mg of D-Luciferin by tail vein injection and were positioned within IVIS® instrument. After 5 min images were taken with 5 min exposure. Radiance represents firefly luciferase-induced luminescence signal intensity measured as photons/second/cm²/steradiant [p/s/cm²/sr]. Statistics: Error bars show SD. Ordinary Two-way ANOVA and Tukey's multiple comparisons test were performed. *p<0.05; **p<0.01, ***p<0.001, ****p<0.00001;

FIG. 2: Biodistribution of rAAV vector genomes determined by qPCR quantification. For determining vector genome content in liver compared to main-off target tissues (LIV=liver; SPL=spleen; LNG=lung; HRT=heart), total DNA was isolated from animals of the above-described in vivo experiment (FIG. 1). Indicated DNA samples were analyzed by relative qPCR quantification using firefly luciferase as target and mHPRT as reference gene. The biodistribution is shown as relative transgene content for the different tissue samples. The target gene contents are defined as cTarget/Reference ratios and represent Target/Reference ratios calibrated by an inter-plate calibrator. Error bars show SD. Mean of rAAV8 relative vector genome content in liver tissue was set as one. Statistics: Error bars show SD. Shown significances relate to rAAV2. One-Way ANOVA und Tukeys Multiple Comparison Test: **p<0.01; ****p<0.0001);

FIG. 3: Transduction of primary human and murine hepatocytes with rMLIV1 and rMLIV3 compared to parental rAAV2 and hepatotropic rAAV8 controls. Primary human (A) or murine (B) hepatocytes were seeded and subsequently transduced with a vector particle per cell ratio (GOI) of 10000. 72 hrs post transduction, cells were lysed and a luciferase assay was performed to determine level of transduction as means of luciferase activity. Luminescence signal shown in relative luminescence units (RLU). Mean RLUs were normalized to protein content measured by Bradford assay. Statistics: Error bars show SD. One-Way ANOVA und Tukeys Multiple Comparison Test: *p≤0.05, **p<0.01; ***p<0.001; ****p<0.0001);

FIG. 4: HCC transduction efficiency of novel AAV2-based capsid variants (rHCCM1, rHCCM2, and rHCCM3) compared to parental rAAV2wt control. A) Representative images of in vivo and in situ imaging of luciferase activity/luminescence signal 3 days post vector injection into the TGFα/c-myc HCC mice. B) Quantification of in situ luciferase activity/luminescence of tumor nodules; Animals received 1.5E11 particles of indicated vectors by tail vein injection. Three days post injection, animals were prepared for in vivo imaging. 5 min after D-luciferin injection (1 mg per animal) a series of images was taken with 3 min of exposure time by IVIS®. One hour after the first measurement, animals received a second injection of D-luciferin. Animals were sacrificed after 5 min, livers were isolated and then imaged with 3 min of exposure time. Counts represent firefly luciferase-induced luminescence signal intensity measured as relative light units (RLU). Signal overlay images (A) generated with a color scale of 50 to 1200 counts. 100 RLU defined as back ground level. Dashed rectangle marks background (BKG) ROI on animal skin; RLU values represent background-corrected signals. Circles represent ROIs marking tumor nodules. Dashed circle mark background (BKG) ROI on non-tissue area; ROI (BKG) tags show BKG-corrected total RLU values measured within ROIs; BKG tags show total RLU values of measured within BKG ROI;

FIG. 5: Transduction of human hepatoma and human hepatocyte cell lines by indicated novel capsid variants compared to parental rAAV2wt control. A) HepG2, Huh7 B) and C) Pop10 were transduced with a particle per cell ratio (GOI) of 1000. 24 hrs post transduction, cells were lysed and luciferase assay was performed. Luciferase activity as luminescence signal is shown in relative luminescence units (RLU). Mean RLU of rAAV2 was set as one. Statistics: One-Way ANOVA und Tukeys Multiple Comparison Test: *p≤0.05, **p<0.01; ***p<0.001; ****p<0.0001);

FIG. 6: Transduction of human hepatoma cell line with indicated capsid variants compared to parental rAAV2wt control. HepG2 were transduced with GOI 1000 and harvested 24 hrs post transduction. Cells were lysed and luciferase assay was performed to detect luciferase activity as luminescence signal shown in relative luminescence units (RLU). Mean RLU of rAAV2 (rAAV2wt control) was set as one. Statistics: One-Way ANOVA und Tukeys Multiple Comparison Test: *p≤0.05, **p<0.01; ***p<0.001; ****p<0.0001).

FIG. 7A-D: VP1 amino acid sequence alignment of AAV serotypes AAV1, AAV2, AAV3b, AAV4, AAV5, AAV6, AAV7, AAV9, AAV10, AAV11 and AAVbovine. I-587 and I-453 represent preferred homologous insertion sites after amino acid positions 587 and 453 (AAV2 VP1 numbering).

### DETAILED DESCRIPTION OF THE PRESENT INVENTION

The present inventors identified new mutant AAV capsid protein or fragment thereof being more efficient and partially more specific in targeting hepatic tissue, hepatocytes, hepatic cells and cell lines or hepatocellular carcinoma (HCC).

The vector particles as well as the capsid proteins are characterized in containing insertions of an insert after at least one of the amino acids having the amino acid numbers 139, 161, 261, 381, 447, 453, 459, 534, 570, 573, 584, 585, 586, 587, 588, 589 of SEQ ID No. 2 corresponding to the capsid protein of AAV, or the homolog capsid protein of the other serotypes of AAV, namely, AAV1, AAV3 to AAV11, and AAV bovine, the insert comprises an oligopeptide of at least four amino acids and at most 30 amino acids, like consisting of five to 12 amino acids, in particular, seven amino acids. The insert may optionally contain further amino acids representing a linker sequence at both sites of said oligopeptide, said linker sequence having independently from one another a size of one to three amino acids, if present.

As used herein, the term “fragment thereof” refers to a protein or a polypeptide derived from the mutated adeno-associated virus capsid protein. Typically, the size of the fragment is at least 50%, like at least 70%, as 80%, 90%, like 95% of the size or length of the sequence it refers to.

The term “mutated AAV capsid protein” means a capsid protein containing an insert composed of amino acids at the distinct positions identified herein referring to the sequence of SEQ ID No. 2 corresponding to the capsid protein VP1 of AAV2.

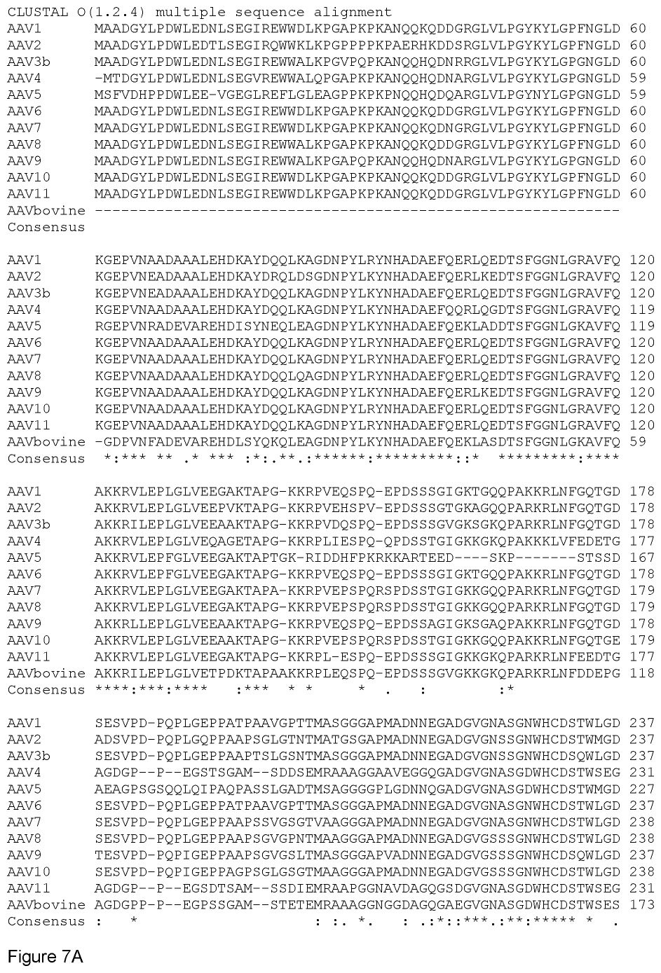

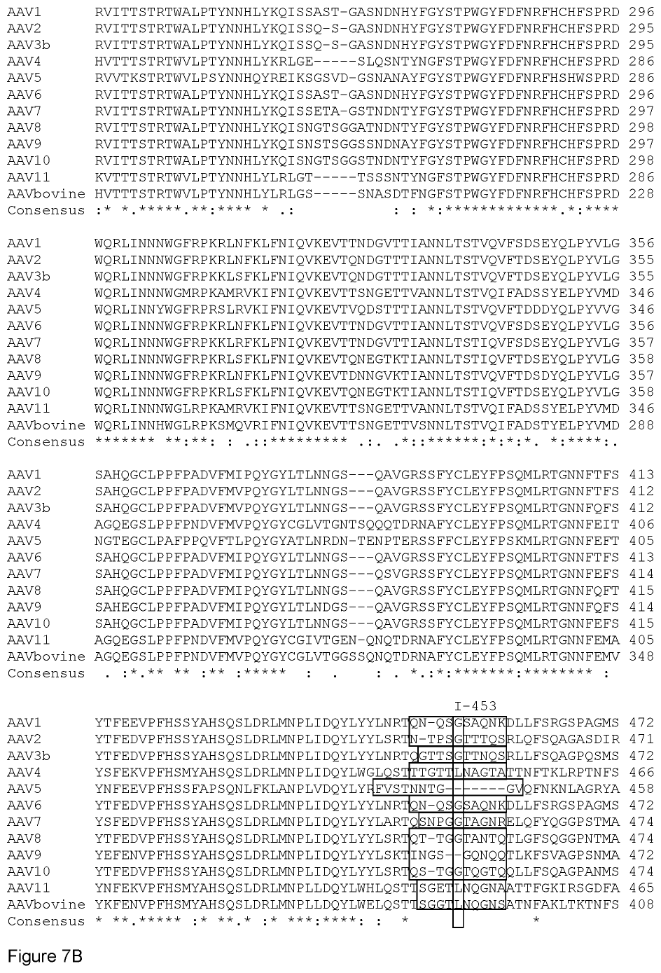

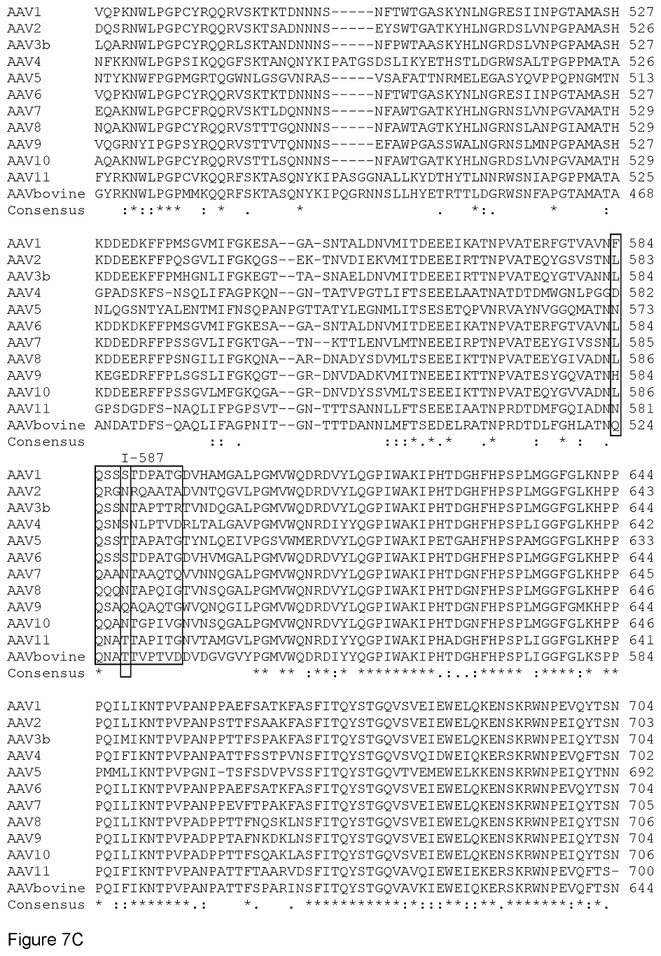

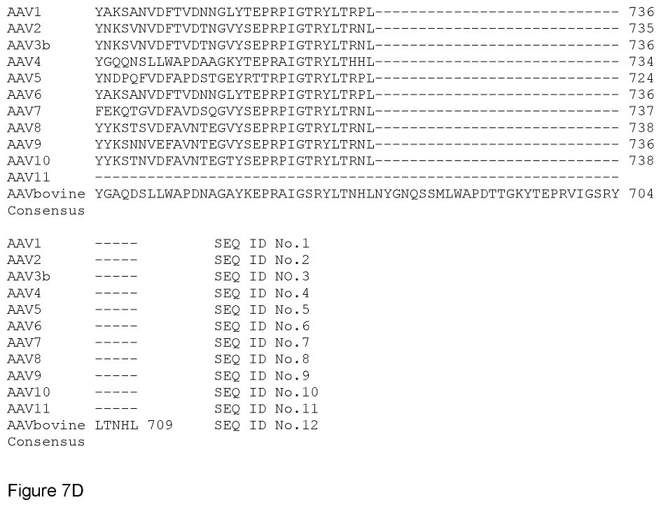

The term “homolog capsid protein” refers to the homolog capsid protein VP1 of the other serotypes of AAV, namely, AAV1 (SEQ ID No. 1), AAV3 (SEQ ID No. 3), AAV4 (SEQ ID No. 4), AAV5 (SEQ ID No. 5), AAV6 (SEQ ID No. 6), AAV7 (SEQ ID No. 7), AAV8 (SEQ ID No. 8, AAV9 (SEQ ID No. 9), AAV10 (SEQ ID No. 10), AAV11 (SEQ ID No. 11) and AAV bovine (SEQ ID No. 12). That is, the homologs are serotype capsid proteins derived from the other AAV serotypes. An overview is given in Vance, M. A. et al., DOI: 10.5772/61988. The serotype alignment is shown in FIG. 7A-D. That is, the homologous insertion sites in other AAV serotypes corresponding to the AAV2 insertion sites can be easily determined by the skilled person,

The term “outer surface of the capsid” identifies the exterior of the capsid, thus, displaying the insert for allowing interaction with an interacting partner. The term “outer surface of the capsid” is used interchangeably with the term “exterior of the capsid”.

The term “linker” refers to an amino acid sequence which is optionally present in the mutated AAV capsid protein according to the present invention. Said linker is a linking element between the oligopeptide present in the insert of the mutated AAV capsid protein and the wild type amino acid sequence of the VP1 protein.

The term “particle” or “AAV particle”, identify a capsid either without DNA or containing DNA. The term “vector particle”, or “vector” identify a capsid containing DNA. The term “AAV” refers to the adeno-associated virus or the derivatives thereof including recombinant AAV vector particles wherein the term “AAV wild type particle” designates the adeno-associated virus capsid as it occurs in nature without DNA or containing DNA. The term “recombinant AAV” or “recombinant vector particle” is an AAV wherein the genome of the virus is substituted with a vector genome, namely, a foreign DNA to be introduced into the cell and is also termed “rAAV”. The term “AAV” or the respective recombinant AAV vector particle and AAV wild type particle include the at least 13 different serotypes known in the art. The AAV of human serotype 2 is also mentioned as AAV2 or AAV2 particle or AAV2 vector particle.

The term “targeting” refers to the improvement of specificity for the target tissues or target cells and/or the improvement of transduction efficiency in the target tissues or target cells by genetic modification of the AAV capsid. The term “targeting molecule” refers to a molecule allowing targeting of AAV particle as described herein to target cells.

The term “target cells” or “target tissue” as used herein refers to specific cell types or tissues representing the target as defined by the targeting molecule present in the mutated AAV capsid proteins or fragments thereof according to the present invention. Namely, the target cells or target tissue is hepatic tissue, hepatocytes, hepatic cells and cell lines, or hepatocellular carcinoma.

The present inventors recognized that inserting the insert at the distinct positions of the capsid protein allows to provide capsid proteins and, eventually, mutated adeno-associated virus particles being more efficient in targeting hepatic tissue, hepatocytes, hepatic cells and cell lines or hepatocellular carcinoma with partially higher specificity.

In an embodiment, the mutated AAV capsid protein, the fragment thereof or a homolog thereof is a mutated AAV capsid protein being derived from AAV type 1, 2, 3b, 4, 5, 6, 7, 8, 9, 10, 11 and bovine. In an embodiment, the mutated AAV capsid protein is derived from the VP1 of serotype AAV2.

In a further embodiment of the present invention, the mutated AAV capsid protein, the fragment thereof or a homolog thereof is a protein wherein the insert is at the position following at least one of the amino acids of 261, 453, 534, 570, 573, 587 and 588. In a preferred embodiment, the position wherein the insert is introduced after positions 453 and 587, respectively. That is, the insertion is in between positions 453 and 454 of SEQ ID No. 2 and/or in between 587 and 588 of SEQ ID No. 2. In an embodiment, the insertion is present in between positions 587 and 588 of SEQ ID No. 2.

The sequence of SEQ ID No. 2 corresponds to the encoding sequence of the VP1 protein of AAV2. In another embodiment, the mutated AAV capsid protein, a fragment thereof, or a homolog thereof according to the present invention is a mutated AAV capsid protein, a fragment thereof, or the homolog thereof comprising at least one further mutation selected from a point mutation, an internal or terminal deletion, a second insertion and a mutation.

For example, the additional mutation may be a mutation described in the art. Further, the additional mutation may be a mutation as described in undisclosed EP 17193742.8.

The mutated AAV capsid protein according to the present invention were obtained by screening the library in vivo in two different orthotopic HCC mouse models following a pre-clearing step for variants that bind heparin since this feature correlates with unspecific binding to a wide variety of different cell types, which express heparan sulfate proteoglycane (HSPG), the AAV2 primary attachment cell surface receptor. One of the HCC mouse models was generated by transplanting a murine hepatoma cell line (Hepa129) into the liver of C3H mice (Schmitz et al, J. Hepatol. 2004. 787-797). A further mouse model represents a transgenic mouse model overexpressing TGF-a and c-myc regulated by ZnCl₂-and albumin-inducible promoters, respectively (Haupenthal J. et al., Neoplasia, 2012, 14(5), 410-419).

The mutated AAV capsid proteins containing the insertion at the predetermined positions as disclosed herein are characterized in displaying the introduced insert from the capsid at the outer surface of the capsid when the capsid is assembled as an AAV particle. That is, the present inventors aim to improve the AAV vector system with regard to efficiency and selectivity of in vivo gene transfer into hepatocytes and its malignant counterpart, hepatocellular carcinoma cells, based on the mutated AAV capsid proteins according to the present invention. That is, superior transduction efficiencies of hepatocytes and HCC have been achieved with higher cell entry rates and more efficient vector genome accessibility for transcription. The variants described herein transduced hepatocytes and HCC more efficiently compared to wild type AAV2 or other known mutated capsid strains. In addition, the mutated AAV capsid proteins according to the present invention are found to be superior regarding expression efficiency, i.e. transcripts per vector copy, arguing for a more efficient intercellular processing again compared to the wild type AAV2.

That is, the displayed insert present in the outer surface of the capsid when the capsid is assembled as an AAV particle increases efficiency of transfection, thus, lower vector doses are required to achieve therapeutic target cell transduction levels in hepatocytes and HCC, respectively.

In an embodiment of the present invention, the mutated AAV capsid protein, the fragment thereof or a homolog thereof according to the present invention are a capsid protein or a protein fragment or homolog thereof wherein the linker sequences in an amino acid sequence is containing or consisting of amino acids G, S and/or A. In a preferred embodiment, the linker consists of at least one of the amino acids A or S, in particular, having amino acids AAA N-terminally and AA C-terminally or ASA N-terminally and AA C-terminally, flanking the amino acid sequence of the oligopeptide inserted at the positions identified herein.

In an embodiment, the insert does not contain any stop codons. That is, it is clear that the mutated AAV capsid protein according to the present invention contains an insert continued with the wild type amino acid present after the amino acid wherein the insert is introduced. In addition, the nucleic acid molecule according to the present invention does not encode a stop codon, thus, stopping translation of the encoded peptide.

In an embodiment, the linker, in particular, the linker mentioned above, are flanking the oligopeptide of the at least five amino acids and at most 12 amino acids, like the seven amino acids described herein.

In an embodiment, the mutated AAV capsid protein, a fragment thereof, or a homolog thereof are a capsid protein, a protein fragment or homolog thereof wherein an oligonucleotide having a size of seven amino acids having a sequence according to any one of SEQ ID No. 13 to 275. In another embodiment, the mutated AAV capsid protein, a fragment thereof, or a homolog thereof are containing the insert having a sequence of SEQ ID No. 276 to 538. The sequences according to any one of SEQ ID No. 13 to 275 are amino acid sequences showing the insert without any linker group while the insert of any one of SEQ ID No. 276 to 538 show the amino acid sequences of suitable insert to be present as inserts in the mutated AAV capsid proteins according to the present invention containing linker sequences at both sites of the peptide sequences shown in SEQ ID Nos. 13 to 275.

That is, an embodiment of the present invention is a protein having an insertion in between 587 and 588 of SEQ ID No. 2 having an oligonucleotide of seven amino acids and a linker sequence at both sides of 1 to 3 amino acids. Preferably the sequence is any one of SEQ ID No. 13 to 21 and 276 to 284.

In a further aspect of the present invention, a mutated adeno-associated virus particle, also named vector particle, comprising the mutated adeno-associated virus capsid protein or a fragment thereof, or a homolog thereof according to the present invention is provided. This AAV particle is more efficient in targeting hepatic tissue, hepatocytes, hepatic cells and cell lines or hepatocellular carcinoma with partially higher specificity. That is, the efficiency of ex vivo or in vivo transduction of the target cell or target tissue as well as the applicability of AAV as a delivery tool is increased by increasing the capacity of the mutated AAV particle to transduce the cell with higher specificity and/or efficiency.

For example, this mutated AAV particle is useful in the manufacture of AAV particle in the manufacture of a medicament for gene therapy. For example, in case of a medicament for gene therapy, the AAV particle according to the present invention carries a functional nucleic acid fragment or a nucleic acid fragment of a molecule of interest. Said molecule of interest includes a nucleic acid fragment encoding a molecule of interest. Being more efficient and having partially higher specificity of targeting the target cells or target tissue, here targeting hepatic tissue, hepatocytes, hepatic cells and cell lines, or HCC, allows to increase efficacy of gene therapy as well as vaccination strategies for preventing or treating malignancies of the mentioned targeted cells and target tissue.

In particular, the mutated AAV particle according to the present invention is suitable for use in targeting hepatocytes and/or HCC.

The mutated AAV particle according to the present invention is particularly useful in the transfer of a gene of interest into hepatocytes or HCC, in particular, for use in gene therapy.

That is, the mutated AAV particles according to the present invention are useful in the expression of therapeutically effective transgenes. For example, metabolic factors, like enzymes may be expressed to overcome defects like genetic defects. In case of e.g. HCC the expression of tumor suppressor genes, suicide genes, pro-inflammatory factors, etc. may be envisaged.

Moreover, the mutated AAV particles are suitable to induce immunologic tolerance against transgenes or antigens introduced into the cells. This is possible particularly in the target cells and target tissue of the liver. Tolerance may help to treat autoimmune disease. In a further aspect, the present invention relates to a nucleic acid molecule encoding the mutated AAV capsid protein or fragment thereof, or a homolog thereof according to the present invention. That is, the nucleic acid molecule according to the present invention is a nucleic acid molecule encoding the AAV capsid protein or fragment thereof as defined herein, for example, containing the oligopeptide of any one of SEQ ID No. 13 to 275 or the insert according to any one of SEQ ID Nos. 276 to 538. Of course, depending on the host cell, a codon optimization may be effected.

A further aspect relates to a nucleic acid vector, in particular, a plasmid comprising the nucleic acid molecule according to the present invention. The skilled person is well-aware of suitable plasmid and vectors. In particular, based on the host cell to be transfected, a plasmid or vector is selected accordingly. Further the nucleic acid vector may contain further genetic AAV helper elements e.g. the rep open reading frame.

In a further aspect, a host cell containing the nucleic acid vector according to the present invention or the nucleic acid molecule according to the present invention is provided.

The host cell may contain further helper plasmids or nucleic acid molecules containing e.g. the adeno-viral helper genes E4, E2A and VA for the production of the recombinant AAV particles. In an embodiment of the present invention, the host cell is a producer for the production of the AAV particle according to the present invention.

In a further aspect, the present invention relates to the mutated AAV particle according to the present invention or the nucleic acid molecule according to the present invention or the nucleic acid vector according to the present invention or the composition as described herein with suitable transgene DNA components. Said transgene DNA components may be known transgene DNA or DNA encoding suitable peptides or proteins accordingly. In another embodiment, a composition is provided comprising the mutated AAV particle according to the present invention, e.g. the composition is a pharmaceutical composition for usage in transducing hepatic tissue, hepatocytes, hepatic cells and cell lines or hepatocellular carcinoma. Further, the present invention relates to the use of the AAV particle according to the present invention, the nucleic acid molecules according to the present invention or the nucleic acid vector according to the present invention in the manufacture of an AAV particle or the manufacture of a medicament for gene therapy.

The mutated AAV capsid protein, a fragment thereof, or a homolog according to the present invention, the mutated AAV particle according to the present invention, the nucleic acid molecule according to the present invention or the nucleic acid vector according to the present invention are particularly useful in targeting hepatocytes and/or HCC as demonstrated herein.

Another aspect of the present invention relates to the mutated AAV capsid protein or a fragment thereof or a homolog thereof according to the present invention, the mutated AAV particle according to the present invention, the nucleic acid molecule according to the present invention or the nucleic acid vector according to the present invention for use in treating diseases involving hepatocytes or for treating HCC: bleeding disorders including Hemophilia A, Hemophilia B, Von Willebrand disease, metabolic disorders including Alpha-1-antitrypsin deficiency, familial hypercholesterolemia, ornithine transcarbamylase deficiency, crigler-najjar syndrome, acute intermittent porphyria, glycogen storage disease type 1 a, liver cancer including hepatocellular carcinoma, hepatoblastoma, collangiocarcinoma, autoimmune disorders including multiple sclerosis, latent autoimmune diabetes, induction of tolerance towards allogenic transplants including liver, kidneys, heart, stem cells, surrogate liver-directed gene therapy including lipoproteinlipase deficiency, diabetes and ornithine transcarbamylase deficiency.

That is, the components described herein including the mutated AAV capsid protein, fragment thereof or homolog thereof, the mutated AAV particle, the nucleic acid molecule or the nucleic acid vector are suitable for use in a method of treating an individual in need thereof. Hence, another aspect relates to a method of treating an individual in need thereof comprising the step of administering a mutated AAV particle according to the present invention or a nucleic acid molecule encoding the mutated AAV virus capsid protein, a fragment thereof, or a homolog thereof according to the present invention, and/or a nucleic acid vector according to the present invention to an individual in need thereof.

Moreover, administration can be conducted by known means. In particular, administration can be conducted by intravenous route, or injection into the (hepatic) portal vein, or intrahepatically, or intratumorally, respectively.

In particular, the method of therapeutic treatment according to the present invention is a method for therapeutic treatment of diseases involving hepatocytes or for treating HCC. Examples include bleeding disorders including Hemophilia A, Hemophilia B, Von Willebrand disease, metabolic disorders including Alpha-1-antitrypsin deficiency, familial hypercholesterolemia, ornithine transcarbamylase deficiency, Crigler-Najjar syndrome, acute intermittent porphyria, glycogen storage disease type 1 a, liver cancer including hepatocellular carcinoma, hepatoblastoma, collangiocarcinoma, autoimmune disorders including multiple sclerosis, latent autoimmune diabetes, induction of tolerance towards allogenic transplants including liver, kidneys, heart, stem cells, surrogate liver-directed gene therapy including lipoproteinlipase deficiency, diabetes and ornithine transcarbamylase deficiency.

In an embodiment of the present invention, the method of prophylactic or therapeutic treatment is a gene therapy including the step of administering the AAV particle according to the present invention or comprising at least one of the mutated AAV capsid protein or fragment thereof, or homolog thereof according to the present invention, a nucleic acid molecule according to the present invention and/or a nucleic acid vector according to the present invention, e.g. in form of a pharmaceutical composition. Said pharmaceutical composition or medicament may contain other suitable components including diluents, excipients or carriers.

Further, a method for ex vivo or in vivo transfer of a gene of interest into hepatocytes or HCC, in particular, by gene therapy, namely, liver directed AAV gene therapy, is disclosed.

The invention will be described further by way of examples without limiting the same thereto.

### EXAMPLES

### Characterization of MLIV Capsid Variants

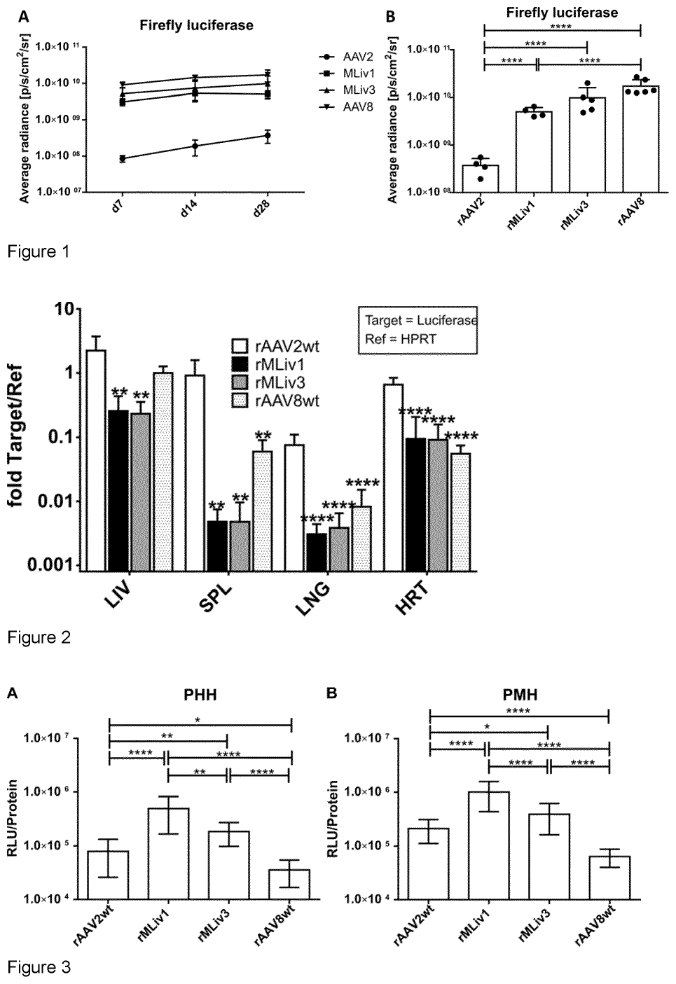

Prior to AAV peptide display library selection the library was pre-cleared from HSPG binding capsid variants by heparin affinity column purification. In the library, the insert is located in between positions 587 and 588 of SEQ ID No 2. Capsid variants that accumulated in liver tissue after an in vivo AAV peptide display library selections in the two different mouse models were named MLIV* library (Hepa129 grafted) and MLIV library (TGF-α/c-myc transgenic), respectively. 92 candidate variants for the grafted and 84 candidate variants for the transgenic mouse model have been identified. The most abundant variants of each of the two libraries were named MLIV1 (MLIV*) (SEQ ID No. 13) and MLIV3 (MLIV) (SEQ ID NO. 14) and characterized in detail. For in vivo evaluation, MLIV1 and MLIV3 were produced as recombinant (r)AAV vectors expressing a luciferase transgene cassette. Transgene expression was controlled by the CMV promoter, a ubiquitously expressed promoter. In detail, 4.8E11 rAAV vector particles were applied to healthy mice via tail vein injection. As control and for comparison, mice received rAAV2 and rAAV8 with natural serotype capsids delivering the same vector genome. 7, 14, and 28 days post injection, mice were injected with D-luciferin (1.5 mg per animal) and the luciferase activity in various body regions was monitored by in vivo imaging using a luminescence signal detecting camera. In all mouse cohorts a significant luciferase activity was detected in the upper abdomen, the liver region, of the animals. The novel capsid variants rMLIV1 and rMLIV3 demonstrated a clear and significant improvement in murine liver transduction efficiency (up to 26-fold) compared to rAAV2 (FIG. 1, A, B). Murine liver transduction efficiency of the new capsid variants is similar to the control vector (rAAV8), which is currently in use in liver-directed human clinical gene therapy trials. Regarding the biodistribution of rMLIV1 and rMLIV3 as determined by qPCR analyses of vector genomes, a clear tropism for murine liver was observed. Specifically, the amount of vector genomes detected in liver was up to 51-fold higher for the novel capsid variants than in spleen, up to 18-fold higher than in the lung, and 3-fold higher than in the heart (FIG. 2). In particular, the observed de-targeting from murine spleen, one of the main off-target tissues of AAV2 (in the murine off-target tissues: rAAV2 genome content>rMLIV capsid variant genome content (182-fold higher in spleen; up to 24-fold higher in lung, 7-fold higher in heart)) (FIG. 2), is an important novel feature of the variants according to the present invention with regard to in vivo gene therapy.

It is well known that the non-human primate serotype rAAV8 shows high transduction efficiency in liver tissue of mice. However, this does not correlate with the transduction efficiency obtained for human liver. In comparison, human serotype rAAV2 vectors are taken up and processed more efficiently in human hepatocytes. To investigate the efficiency of the newly developed capsid variants in the context of human hepatic tissue, primary human hepatocytes were transduced with rMLIV1 and rMLIV3 (and the controls rAAV2 and rAAV8). In addition, the same vectors were assayed on primary murine hepatocytes (GOI 1E4 rAAV vector particles per cell, 72 hrs). For quantification of luciferase expression, luciferase activity was measured in cell lysates by adding luciferin followed by luminescent signal detection (Promega Luciferase Assay kit). Here, rAAV2 and the novel capsid variants rMLIV1 and rMLIV3 were clearly superior to rAAV8, which is used in human clinical trials (Nathwani 2014 doi: 10.1056/NEJMoa1407309; Pierce 2018, doi: 10.1111/hae.13489) (FIG. 3, A and B).

Due to the strong liver tropism across species and high hepatic transduction efficiency, MLIV1 and MLIV3 represent promising new developments for in vivo gene therapy. Furthermore, the significantly higher efficiency compared to rAAV8 in primary human hepatocytes which have been observed following ex vivo transduction and which is currently confirmed in humanized mice are an excellent basis for moving these novel vectors forward as tools for liver-directed human clinical gene therapy trials.

### Characterization of HCCM Capsid Variants

Prior to AAV peptide display library selection the library was pre-cleared from HSPG binding capsid variants by heparin affinity column purification. Capsid variants that accumulated most abundantly in HCC tissue after in vivo AAV peptide display library selections in the two different mouse models were named HCCM* (selected in Hepa129 grafted mouse model) and HCCM (selected in TGF-α/c-myc transgenic mouse model). For the HCCM* selection route, before in vivo selection, the AAV peptide display library was pe-selected in vitro on target cells Hepa129. We identified 103 candidate variants in the grafted and 89 candidate variants in the transgenic mouse model. These capsid variants were further scored with regard to enrichment in HCC (target) compared to liver (main off-target) tissue. The best scored capsid variants were ranked regarding enrichment in HCC compared to all analyzed off-target tissues (liver, heart, spleen, skeleton muscle, kidney, and pancreas). The best scored capsid variants were named HCCM1 (SEQ ID No.15), HCCM2 (SEQ ID No.16), and HCCM3 (SEQ ID No.17) (HCCM library) and HCCM*1 (SEQ ID No.18), HCCM*2 (SEQ ID No.19), HCCM*3 (SEQ ID No.20), and HCCM*4 (SEQ ID No.21) (HCCM* library). They were produced as rAAV vectors expressing a luciferase reporter transgene cassette for characterization. Expression of the transgene was controlled by the SFFV promoter.

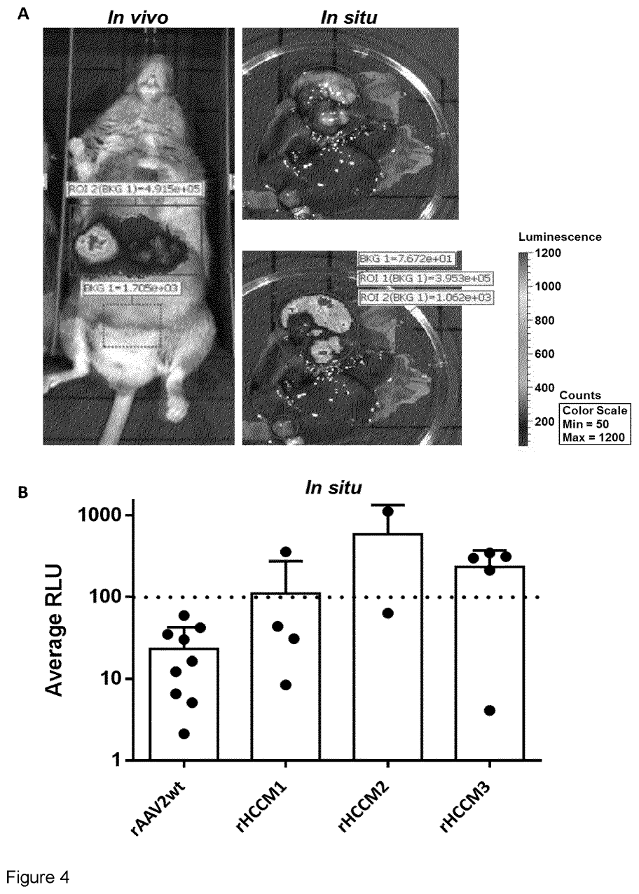

HCCM1, HCCM2, and HCCM3 were administered to TGF-a/c-myc mice via tail vein injection (1.5E11 rAAV vector particles per animal). For comparison the parental AAV (rAAV2 vector with natural occurring serotype delivering the same vector genome) was applied. 3 days post injection, mice were injected with D-luciferin (1 mg per animal) and after 5 min luciferase activity in the different body regions was measured by in vivo imaging analysis using a luminescence signal detecting camera (3 min exposure). In addition, from all animals tumor bearing livers were isolated 5 min after repeated administration of luciferin and the luminescence signal was determined in situ (3 min exposure). Exclusively, mouse cohorts treated with the novel rHCCM capsid variants demonstrated significant luciferase activity in the tumors, whereas in the rAAV2 cohort luminescence signals remained below background level (100 RLU) (FIG. 4, A, B). The novel capsid variants rHCCM1, rHCCM2, and rHCCM3 demonstrated a clear improvement in HCC transduction efficiency compared to control.

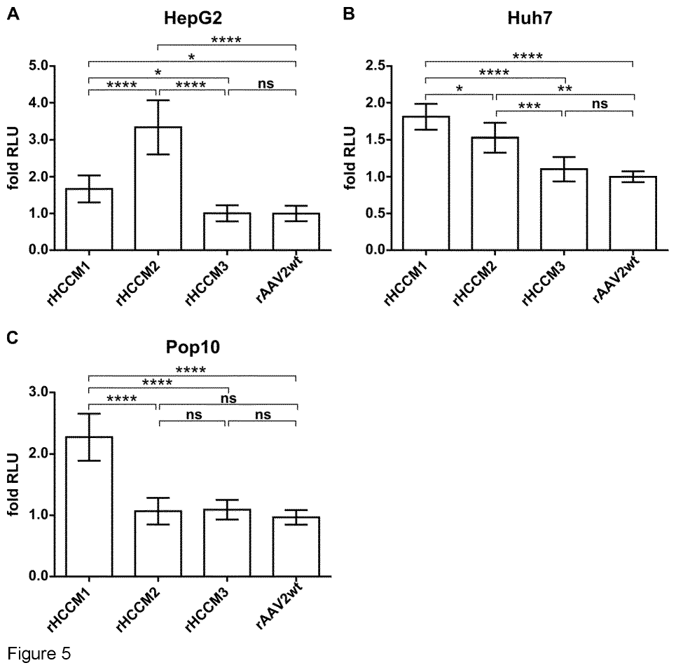

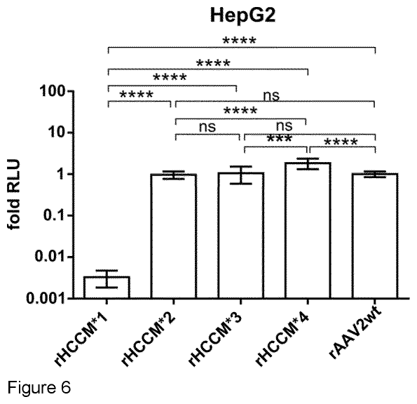

rAAV2 is very efficient in transducing human hepatoma and hepatocytes cell lines (GOI of 1000 AAV vector particles per cell, 24 hrs). To estimate the efficiency of the newly developed HCCM and HCCM* capsid variants in the context of human hepatic tissue, several human hepatoma/hepatocyte cell lines were transduced with the newly developed capsid variants rHCCM1, rHCCM2, rHCCM3, rHCCM*1, rHCCM*2, rHCCM*3, and rHCCM*4 as well as the parental control (rAAV2). rAAV vectors expressed Renilla luciferase controlled by CMV promoter. Transgene expression level was quantified by luciferase assay (Promega Renilla Luciferase Assay kit). The novel capsid variants rHCCM1, rHCCM2, rHCCM3 (FIG. 5, A to B), rHCCM*2, rHCCM*3, and rHCCM*4 (FIG. 6) demonstrated transduction efficiencies comparable or even superior to rAAV2 in these human hepatoma cell lines (FIG. 5, A and B).

To date, treatment of HCC per se and thus also AAV-based gene therapy targeting HCC is limited by low efficiency. The novel HCCM capsid variants show clearly improved transduction of HCC nodules following intravenous injection and thus represent promising candidates for novel treatment strategies employing the AAV vector system.

1. A mutated adeno-associated virus (AAV) capsid protein or a fragment thereof wherein an insert is inserted after at least one the amino acids having the amino acid numbers 139, 161, 261, 381, 447, 453, 459, 534, 570, 573, 584, 585, 586, 587, 588, 589 of SEQ ID No. 2 corresponding to the capsid protein of AAV type 2 or the homolog capsid protein of the other serotypes of AAV, namely, AAV1, AAV3b, AAV4, AAV5, AAV6, AAV7, AAV8, AAV9, AAV10, AAV11, and AAV bovine, the insert comprises an oligopeptide of at least four amino acids and at most thirty amino acids, like consisting of five to twelve amino acids, in particular, seven amino acids, said insert may optionally has further amino acids representing a linker sequence at both sites of said oligopeptide, said linker sequence having independently from one another a size of 1 to 3 amino acids, if present; wherein the mutated AAV capsid protein or a fragment thereof or its homologs being more efficient in targeting hepatic tissue, hepatocytes, hepatic cells and cell lines or hepatocellular carcinoma (HCC) with higher specificity.

2. The mutated AAV capsid protein, a fragment thereof or a homolog thereof according to claim 1 wherein the AAV is type 1, 2, 3b, 5, 6, 7, 8, 9, 10, 4, 11, and bovine.

3. The mutated AAV capsid protein, a fragment thereof or a homolog thereof according to claim 1 wherein the insert are at the positions following at least one of the amino acids of 261, 453, 534, 570, 573, 587 and 588.

4. The mutated AAV capsid protein, a fragment thereof or a homolog thereof according to claim 1 wherein the insert is inserted after aa 587 of SEQ ID. No. 2 having an oligonucleotide of seven amino acids and a linker sequence at both sides of from 1 to 3 amino acids.

5. The mutated AAV-capsid protein, a fragment thereof or a homolog thereof according to claim 1 wherein the insert is a sequence of SEQ. ID. No. 13 to 21 and 276 to 284.

6. The mutated AAV capsid protein, a fragment thereof or a homolog thereof according to claim 1 wherein the mutated AAV capsid protein, the fragment thereof, or the homolog thereof comprise at least one further mutation selected from a point mutation, an internal or terminal deletion, a second insertion and a substitution.

7. The mutated AAV capsid protein, a fragment thereof, or a homolog thereof according to claim 1 when the linker sequence is an amino acid sequence containing or consisting of the amino acids G, S and/or A, in particular, wherein the linker consists of at least one of amino acids A or S, in particular, having amino acids AAA N-terminally and AA C-terminally, or ASA N-terminally and AA C-terminally, flanking the oligopeptide of at least four to at most thirty amino acids, like at least five amino acids and at most twelve amino acids, like seven amino acids.

8. The mutated AAV capsid protein, a fragment thereof, or a homolog thereof according to claim 1 wherein the oligopeptide having a size of seven amino acids is a sequence according to any one of SEQ ID No. 13 to 275.

9. The mutated AAV capsid protein, a fragment thereof, or a homolog thereof according to claim 1 wherein the insert is any one of SEQ ID No. 276 to 538.

10. A mutated adeno-associated virus (AAV) particle comprising the mutated AAV capsid protein, a fragment thereof or a homolog thereof according to claim 1.

11. A nucleic acid molecule encoding the mutated AAV capsid protein, a fragment thereof or a homolog thereof according to claim 1.

12. A nucleic acid vector comprising the nucleic acid molecule according to claim 11.

13. A host cell containing the nucleic acid vector according to claim 12.

14. A method of manufacturing a medicament for gene therapy by using the AAV particle according to claim 10.

15. A method of transducing hepatocytes and/or hepatocellular carcinoma (HCC) in vivo or ex vivo by exposing the hepatocytes and/or HCC to the mutated AAV capsid protein, a fragment thereof or a homolog according to claim 1.

16. A method of treating diseases involving hepatocytes in a subject selected from the group consisting of bleeding disorders including Hemophilia A, Hemophilia B, Von Willebrand disease, metabolic disorders including Alpha-1-antitrypsin deficiency, familial hypercholesterolemia, ornithine transcarbamylase deficiency, crigler-najjar syndrome, acute intermittent porphyria, glycogen storage disease type la, liver cancer including hepatocellular carcinoma, hepatoblastoma, collangiocarcinoma, autoimmune disorders including multiple sclerosis, latent autoimmune diabetes, induction of tolerance towards allogenic transplants including liver, kidneys, heart, stem cells, surrogate liver-directed gene therapy including lipoproteinlipase deficiency, diabetes and ornithine transcarbamylase deficiency; or for treating HCC comprising providing the subject with a mutated AAV capsid protein, fragment thereof or a homolog according to claim 1.

17. A method for transferring a gene of interest into hepatocytes and/or HCC by exposing the hepatocytes and/or HCC to the mutated AAV capsid protein, a fragment thereof or a homolog according to claim 1.

18. A host cell containing a nucleic acid molecule according to claim 11.

19. A method of transducing hepatocytes and/or hepatocellular carcinoma (HCC) in vivo or ex vivo by exposing the hepatocytes or HCC to the nucleic acid vector of claim 12.

20. A method for transferring a gene of interest into hepatocytes and/or HCC by exposing the hepatocytes and/or HCC to the nucleic acid vector of claim 17.

21. A method of manufacturing a medicament for gene therapy by using the nucleic acid according to claim 11 in the manufacture of the medicament.

22. A method of manufacturing a medicament for gene therapy by using the nucleic acid vector according to claim 12 in the manufacture of the medicament.

23. A method of treating diseases involving hepatocytes in a subject selected from the group consisting of bleeding disorders including Hemophilia A, Hemophilia B, Von Willebrand disease, metabolic disorders including Alpha-1-antitrypsin deficiency, familial hypercholesterolemia, ornithine transcarbamylase deficiency, crigler-najjar syndrome, acute intermittent porphyria, glycogen storage disease type la, liver cancer including hepatocellular carcinoma, hepatoblastoma, collangiocarcinoma, autoimmune disorders including multiple sclerosis, latent autoimmune diabetes, induction of tolerance towards allogenic transplants including liver, kidneys, heart, stem cells, surrogate liver-directed gene therapy including lipoproteinlipase deficiency, diabetes and ornithine transcarbamylase deficiency; or for treating HCC comprising providing the subject with a mutated AAV particle according to claim 10.

24. A method of treating diseases involving hepatocytes in a subject selected from the group consisting of bleeding disorders including Hemophilia A, Hemophilia B, Von Willebrand disease, metabolic disorders including Alpha-1-antitrypsin deficiency, familial hypercholesterolemia, ornithine transcarbamylase deficiency, crigler-najjar syndrome, acute intermittent porphyria, glycogen storage disease type la, liver cancer including hepatocellular carcinoma, hepatoblastoma, collangiocarcinoma, autoimmune disorders including multiple sclerosis, latent autoimmune diabetes, induction of tolerance towards allogenic transplants including liver, kidneys, heart, stem cells, surrogate liver-directed gene therapy including lipoproteinlipase deficiency, diabetes and ornithine transcarbamylase deficiency; or for treating HCC comprising providing the subject with the nucleic acid vector according to claim 12.
